SEQUENCE LISTING (27 sequences)

SEQ ID NO: 1 - protein, 744 aa, synthetic construct
  source 1..744: note = DIA-19 sequence
QITLKESGPTLVKPTQTLTLTCTFSGFSLSTSGVGVAWIRQPPGKALEWLALIYWDNDKRSSPSLNNRLTITKDTSKNQVVLTMTNMDPEDTATYYCAHFFSHYDSSNYYYGSWFDPWGQGTLVTVSSGGGGSGGGGSDIQMTQSPSSLSASVGDRVTITCRASQSISNYLNWYQQKPGKAPKLLIYVASSLQSGVPSRFSGSGSGTDFTLTISSLQPEDFATYYCQQSYSTPFTFGPGTKVDIKGGGGSGGGGSGGGGSGGGGSGGGGSEVQLVESGGGVVQPGGSLRLSCAVSGFTFDDYAMHWVRQAPGKGLDWVSLISGDGSYTYYADSVKGRFTISRDSSKNSLYLQMNSLRTEDTALYYCAKAQTPTLWWLQDAFDIWGQGTMVTVSSGGGGSGGGGSEIVLTQSPGTLSLSPGERATLSCRASQSFDSRYLGWYQQKSGQAPRLLIYGASSRATGIPDRFSGSGSGTDFTLTISRLEPEDFAVYYCQQFGDSPFTFGQGTKLEIKGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 744 aa, synthetic construct
  source 1..744: note = DIA-20 sequence
EIVLTQSPGTLSLSPGERATLSCRASQSFDSRYLGWYQQKSGQAPRLLIYGASSRATGIPDRFSGSGSGTDFTLTISRLEPEDFAVYYCQQFGDSPFTFGQGTKLEIKGGGGSGGGGSEVQLVESGGGVVQPGGSLRLSCAVSGFTFDDYAMHWVRQAPGKGLDWVSLISGDGSYTYYADSVKGRFTISRDSSKNSLYLQMNSLRTEDTALYYCAKAQTPTLWWLQDAFDIWGQGTMVTVSSGGGGSGGGGSGGGGSGGGGSGGGGSDIQMTQSPSSLSASVGDRVTITCRASQSISNYLNWYQQKPGKAPKLLIYVASSLQSGVPSRFSGSGSGTDFTLTISSLQPEDFATYYCQQSYSTPFTFGPGTKVDIKGGGGSGGGGSQITLKESGPTLVKPTQTLTLTCTFSGFSLSTSGVGVAWIRQPPGKALEWLALIYWDNDKRSSPSLNNRLTITKDTSKNQVVLTMTNMDPEDTATYYCAHFFSHYDSSNYYYGSWFDPWGQGTLVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 744 aa, synthetic construct
  source 1..744: note = DIA-21 sequence
EVQLVESGGGVVQPGGSLRLSCAVSGFTFDDYAMHWVRQAPGKGLDWVSLISGDGSYTYYADSVKGRFTISRDSSKNSLYLQMNSLRTEDTALYYCAKAQTPTLWWLQDAFDIWGQGTMVTVSSGGGGSGGGGSEIVLTQSPGTLSLSPGERATLSCRASQSFDSRYLGWYQQKSGQAPRLLIYGASSRATGIPDRFSGSGSGTDFTLTISRLEPEDFAVYYCQQFGDSPFTFGQGTKLEIKGGGGSGGGGSGGGGSGGGGSGGGGSQITLKESGPTLVKPTQTLTLTCTFSGFSLSTSGVGVAWIRQPPGKALEWLALIYWDNDKRSSPSLNNRLTITKDTSKNQVVLTMTNMDPEDTATYYCAHFFSHYDSSNYYYGSWFDPWGQGTLVTVSSGGGGSGGGGSDIQMTQSPSSLSASVGDRVTITCRASQSISNYLNWYQQKPGKAPKLLIYVASSLQSGVPSRFSGSGSGTDFTLTISSLQPEDFATYYCQQSYSTPFTFGPGTKVDIKGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 744 aa, synthetic construct
  source 1..744: note = DIA-22 sequence
DIQMTQSPSSLSASVGDRVTITCRASQSISNYLNWYQQKPGKAPKLLIYVASSLQSGVPSRFSGSGSGTDFTLTISSLQPEDFATYYCQQSYSTPFTFGPGTKVDIKGGGGSGGGGSQITLKESGPTLVKPTQTLTLTCTFSGFSLSTSGVGVAWIRQPPGKALEWLALIYWDNDKRSSPSLNNRLTITKDTSKNQVVLTMTNMDPEDTATYYCAHFFSHYDSSNYYYGSWFDPWGQGTLVTVSSGGGGSGGGGSGGGGSGGGGSGGGGSEIVLTQSPGTLSLSPGERATLSCRASQSFDSRYLGWYQQKSGQAPRLLIYGASSRATGIPDRFSGSGSGTDFTLTISRLEPEDFAVYYCQQFGDSPFTFGQGTKLEIKGGGGSGGGGSEVQLVESGGGVVQPGGSLRLSCAVSGFTFDDYAMHWVRQAPGKGLDWVSLISGDGSYTYYADSVKGRFTISRDSSKNSLYLQMNSLRTEDTALYYCAKAQTPTLWWLQDAFDIWGQGTMVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 215 aa, synthetic construct
  source 1..215: note = CoV56 light chain amino acid sequence
DIQMTQSPSSLSASVGDRVTITCRASQSISNYLNWYQQKPGKAPKLLIYVASSLQSGVPSRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECS

SEQ ID NO: 6 - protein, 454 aa, synthetic construct
  source 1..454: note = CoV56 heavy chain amino acid sequence
EVQLVESGGGVVQPGGSLRLSCAVSGFTFDDYAMHWVRQAPGKGLDWVSLISGDGSYTYYADSVKGRFTISRDSSKNSLYLQMNSLRTEDTALYYCAKAQTPTLWWLQDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 216 aa, synthetic construct
  source 1..216: note = L4.65 light chain amino acid sequence
EIVLTQSPGTLSLSPGERATLSCRASQSFDSRYLGWYQQKSGQAPRLLIYGASSRATGIPDRFSGSGSGTDFTLTISRLEPEDFAVYYCQQFGDSPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECS

SEQ ID NO: 8 - protein, 458 aa, synthetic construct
  source 1..458: note = L4.65 heavy chain amino acid sequence
QITLKESGPTLVKPTQTLTLTCTFSGFSLSTSGVGVAWIRQPPGKALEWLALIYWDNDKRSSPSLNNRLTITKDTSKNQVVLTMTNMDPEDTATYYCAHFFSHYDSSNYYYGSWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 215 aa, synthetic construct
  source 1..215: note = LY-CoV1404 light chain amino acid sequence
QSALTQPASVSGSPGQSITISCTATSSDVGDYNYVSWYQQHPGKAPKLMIFEVSDRPSGISNRFSGSKSGNTASLTISGLQAEDEADYYCSSYTTSSAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 449 aa, synthetic construct
  source 1..449: note = LY-CoV1404 heavy chain amino acid sequence
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 7 aa, synthetic construct
  source 1..7: note = 65 mAb HCDR1
TSGVGVA

SEQ ID NO: 12 - protein, 16 aa, synthetic construct
  source 1..16: note = 65 mAb HCDR2
LIYWDNDKRSSPSLNN

SEQ ID NO: 13 - protein, 18 aa, synthetic construct
  source 1..18: note = 65 mAb HCDR3
FFSHYDSSNYYYGSWFDP

SEQ ID NO: 14 - protein, 12 aa, synthetic construct
  source 1..12: note = 65 mAb LCDR1
RASQSFDSRYLG

SEQ ID NO: 15 - protein, 7 aa, synthetic construct
  source 1..7: note = 65 mAb LCDR2
GASSRAT

SEQ ID NO: 16 - protein, 9 aa, synthetic construct
  source 1..9: note = 65 mAb LCDR3
QQFGDSPFT

SEQ ID NO: 17 - protein, 5 aa, synthetic construct
  source 1..5: note = 56 mAb HCDR1
DYAMH

SEQ ID NO: 18 - protein, 17 aa, synthetic construct
  source 1..17: note = 56 mAb HCDR2
LISGDGSYTYYADSVKG

SEQ ID NO: 19 - protein, 15 aa, synthetic construct
  source 1..15: note = 56 mAb HCDR3
AQTPTLWWLQDAFDI

SEQ ID NO: 20 - protein, 11 aa, synthetic construct
  source 1..11: note = 56 mAb LCDR1
RASQSISNYLN

SEQ ID NO: 21 - protein, 7 aa, synthetic construct
  source 1..7: note = 56 mAb LCDR2
VASSLQS

SEQ ID NO: 22 - protein, 9 aa, synthetic construct
  source 1..9: note = 56 mAb LCDR3
QQSYSTPFT

SEQ ID NO: 23 - protein, 128 aa, synthetic construct
  source 1..128: note = 65 mAb VH
QITLKESGPTLVKPTQTLTLTCTFSGFSLSTSGVGVAWIRQPPGKALEWLALIYWDNDKRSSPSLNNRLTITKDTSKNQVVLTMTNMDPEDTATYYCAHFFSHYDSSNYYYGSWFDPWGQGTLVTVSS

SEQ ID NO: 24 - protein, 108 aa, synthetic construct
  source 1..108: note = 65 mAb VL
EIVLTQSPGTLSLSPGERATLSCRASQSFDSRYLGWYQQKSGQAPRLLIYGASSRATGIPDRFSGSGSGTDFTLTISRLEPEDFAVYYCQQFGDSPFTFGQGTKLEIK

SEQ ID NO: 25 - protein, 124 aa, synthetic construct
  source 1..124: note = 56 mAb VH
EVQLVESGGGVVQPGGSLRLSCAVSGFTFDDYAMHWVRQAPGKGLDWVSLISGDGSYTYYADSVKGRFTISRDSSKNSLYLQMNSLRTEDTALYYCAKAQTPTLWWLQDAFDIWGQGTMVTVSS

SEQ ID NO: 26 - protein, 107 aa, synthetic construct
  source 1..107: note = 56 mAb VL
DIQMTQSPSSLSASVGDRVTITCRASQSISNYLNWYQQKPGKAPKLLIYVASSLQSGVPSRFSGSGSGTDFTLTISSLQPEDFATYYCQQSYSTPFTFGPGTKVDIK

SEQ ID NO: 27 - protein, 744 aa, synthetic construct
  source 1..744: note = DIA-23 sequence
QITLKESGPTLVKPTQTLTLTCTFSGFSLSTSGVGVAWIRQPPGKALEWLALIYWDNDKRSSPSLNNRLTITKDTSKNQVVLTMTNMDPEDTATYYCAHFFSHYDSSNYYYGSWFDPWGQGTLVTVSSGGGGSGGGGSDIQMTQSPSSLSASVGDRVTITCRASQSISNYLNWYQQKPGKAPKLLIYVASSLQSGVPSRFSGSGSGTDFTLTISSLQPEDFATYYCQQSYSTPFTFGPGTKVDIKGGGGSGGGGSGGGGSGGGGSGGGGSEVQLVESGGGVVQPGGSLRLSCAVSGFTFDDYAMHWVRQAPGKGLDWVSLISGDGSYTYYADSVKGRFTISRDSSKNSLYLQMNSLRTEDTALYYCAKAQTPTLWWLQDAFDIWGQGTMVTVSSGGGGSGGGGSEIVLTQSPGTLSLSPGERATLSCRASQSFDSRYLGWYQQKSGQAPRLLIYGASSRATGIPDRFSGSGSGTDFTLTISRLEPEDFAVYYCQQFGDSPFTFGQGTKLEIKGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0242448 A1
(43) Pub. Date: Aug. 20, 2026

(54) BROAD-SPECTRUM BISPECIFIC ANTIBODY AGAINST NOVEL CORONAVIRUSES

(71) Applicants: INSTITUTE OF MICROBIOLOGY, CHINESE ACADEMY OF SCIENCES, Beijing (CN); SHENYANG SUNSHINE PHARMACEUTICAL CO., LTD., Shenyang (CN); SHANXI ACADEMY OF ADVANCED RESEARCH AND INNOVATION, Taiyuan (CN); CAPITAL MEDICAL UNIVERSITY, Beijing (CN); TIANJIN INSTITUTE OF INDUSTRIAL BIOTECHNOLOGY, CHINESE ACADEMY OF SCIENCES, Tianjin (CN)

(72) Inventors: Fu GAO, Beijing (CN); Yan WU, Beijing (CN); Lianpan DAI, Beijing (CN); Feng GAO, Tianjin (CN); Zhou TONG, Beijing (CN); Qingwei CUI, Taiyuan (CN); Haomin HUANG, Shenyang (CN); Lan DENG, Shenyang (CN)

(73) Assignees: INSTITUTE OF MICROBIOLOGY, CHINESE ACADEMY OF SCIENCES, Beijing (CN); SHENYANG SUNSHINE PHARMACEUTICAL CO., LTD., Shenyang (CN); SHANXI ACADEMY OF ADVANCED RESEARCH AND INNOVATION, Taiyuan (CN); CAPITAL MEDICAL UNIVERSITY, Beijing (CN); TIANJIN INSTITUTE OF INDUSTRIAL BIOTECHNOLOGY, CHINESE ACADEMY OF SCIENCES, Tianjin (CN)

(21) Appl. No.: 19/158,888

(22) PCT Filed: Nov. 13, 2023
(86) PCT No.: PCT/CN2023/131373

§ 371 Date: Aug. 22, 2025

(30) Foreign Application Priority Data
Feb. 24, 2023 (CN) 2023101640748

(51) Int. Cl. C07K 16/104; A61K 39/00; A61P 31/14; G01N 33/569

(52) U.S. Cl. CPC C07K 16/104; A61P 31/14; G01N 33/56983; A61K 2039/505; C07K 2317/31; C07K 2317/35; C07K 2317/565; C07K 2317/76; C07K 2317/92; G01N 2333/165

(57) ABSTRACT

The present disclosure provides a broad-spectrum bispecific antibody against novel coronaviruses, comprising a first targeting domain D1 targeting a first epitope of a SARS-CoV-2 RBD domain, and a second targeting domain D2 targeting a second epitope of the SARS-CoV-2 RBD domain. The bispecific antibody of the present disclosure has a broad-spectrum neutralizing activity and can effectively neutralize SARS-CoV-2 and various highly infectious and harmful SARS-CoV-2 variant strains, thus showing significant application value in the prevention, treatment, and/or detection of infections caused by novel coronaviruses.

### FIELD OF TECHNOLOGY

The present disclosure relates to the technical field of fusion proteins, and more particularly, relates to a broad-spectrum bispecific antibody against novel coronaviruses.

### BACKGROUND

According to the latest data from the World Health Organization (WHO), the novel coronavirus (SARS-CoV-2) seriously endangers the physical and mental health and life safety of humans. People of all ages are at risk of contracting SARS-CoV-2 and its serious diseases; and those who are aged 60 years, who live in nursing homes or long-term care facilities, and who have chronic illnesses have a higher likelihood of developing severe COVID-19 diseases. At present, Omicron variant strains have become the dominant strains across the world due to their greater transmission capacity, faster transmission, lower infectious dose and greater immune escape, and they are also constantly mutating into new subvariant strains that exhibit significant resistance to a number of previously approved vaccines and monoclonal antibodies intended for prevention and treatment. Among the current neutralizing antibodies that have been on the market worldwide, only Eli Lilly's LY-CoV1404 still shows protective efficacy against BA.5, but it is no longer effective against the predominantly prevalent BQ.1.1 and XBB.

According to the mutation trend of novel coronaviruses, there is an urgent need for protective antibodies against novel mutant viruses. In particular, it is necessary to find antibodies capable of targeting the conserved regions of viruses (i.e., regions that are less susceptible to mutations), such that the antibodies may achieve a strong ability to protect against the risk of viral evolution (mutation). Due to the single binding site, monoclonal antibodies show a highly uncertain ability to resist viral re-mutation, posing a greater risk. Bispecific antibodies, however, can have more non-overlapping binding sites and thus target different regions of the virus. Even if the virus has one or two more mutation sites, the affinity and neutralizing activity of the bispecific antibodies for the virus will not be affected, which further reduces the risk of viral re-mutation.

Therefore, there is an urgent need in the art to develop a broad-spectrum bispecific antibody against novel coronaviruses.

### SUMMARY

The present disclosure provides a broad-spectrum bispecific antibody against novel coronaviruses.

In a first aspect, the present disclosure provides a bispecific antibody against novel coronaviruses, comprising: a first targeting domain D1, which targets a first epitope of a SARS-CoV-2 RBD domain; and a second targeting domain D2, which targets a second epitope of the SARS-CoV-2 RBD domain; wherein the first epitope of the SARS-CoV-2 RBD domain has one or more residues selected from the following residues in an RBD region of an S protein: 346R, 437N, 438S, 439N, 440N, 441L, 443S, 444K, 445V, 446G, 447G, 448N, 449Y, 450N, 498Q, 499P, 500T, and 506Q; and the second epitope of the SARS-CoV-2 RBD domain has one or more residues selected from the following residues in the RBD region of an S protein: 352A, 353W, 355R, 357R, 393T, 394N, 396Y, 462K, 463P, 464F, 465E, 466R, 468I, 516E, 518L, 519H, and 520A.

In some embodiments, the first epitope has 1 to n residues selected from the following positions in an RBD region of an S protein: 346R, 437N, 438S, 439N, 440N, 441L, 443S, 444K, 445V, 446G, 447G, 448N, 449Y, 450N, 498Q, 499P, 500T, and 506Q, with n selected from 2, 3, 4, 5, 6, 7, 8, 9, 10, 11, 12, 13, 14, 15, 16, 17 or 18.

In some embodiments, the second epitope has 1 to m residues selected from the following positions in the RBD region of the S protein: 352A, 353W, 355R, 357R, 393T, 394N, 396Y, 462K, 463P, 464F, 465E, 466R, 468I, 516E, 518L, 519H, and 520A, with m selected from 2, 3, 4, 5, 6, 7, 8, 9, 10, 11, 12, 13, 14, 15, 16 or 17.

In some embodiments, the first epitope and the second epitope do not overlap or substantially do not overlap.

In some embodiments, D1 and D2 are each independently selected from: Fab, Fab′, F(ab′)2, an Fv fragment, a single-chain Fv (scFv) fragment and a single-domain fragment, preferably a single-chain Fv (scFv), an Fv fragment or a Fab fragment.

In some embodiments, D1 and D2 are linked in series or parallel, preferably in series.

In some embodiments, the bispecific antibody further comprises an optional third targeting binding domain D3.

In some embodiments, the third targeting binding domain D3 targets a third epitope of the SARS-CoV-2 RBD domain.

In some embodiments, the third targeting binding domain D3 is selected from: Fab, Fab′, F(ab′)2, an Fv fragment, a single-chain Fv (scFv) fragment and a single-domain fragment, preferably a single-chain Fv (scFv), an Fv fragment or a Fab fragment.

In some embodiments, the first targeting domain D1 comprises a first heavy chain variable region and a first light chain variable region, wherein the first heavy chain variable region comprises a HCDR1 of SEQ ID NO: 11, a HCDR2 of SEQ ID NO: 12, and a HCDR3 of SEQ ID NO: 13; and the first light chain variable region comprises a LCDR1 of SEQ ID NO: 14, a LCDR2 of SEQ ID NO: 15, and a LCDR3 of SEQ ID NO: 16.

In some embodiments, the first heavy chain variable region comprises or consists of an amino acid sequence of SEQ ID NO: 23 or an amino acid sequence having 95%, 96%, 97%, 98% or 99% sequence identity to the amino acid sequence of SEQ ID NO: 23 (with unchanged or substantially unchanged CDRs); and/or the first light chain variable region comprises or consists of an amino acid sequence of SEQ ID NO: 24, or an amino acid sequence having 95%, 96%, 97%, 98% or 99% sequence identity to the amino acid sequence of SEQ ID NO: 24 (with unchanged or substantially unchanged CDRs).

In some embodiments, the first heavy chain variable region has an amino acid sequence of SEQ ID NO: 23; and the first light chain variable region has an amino acid sequence of SEQ ID NO: 24.

In some embodiments, the second targeting domain D2 comprises a second heavy chain variable region and a second light chain variable region, wherein the second heavy chain variable region comprises a HCDR1 of SEQ ID NO: 17, a HCDR2 of SEQ ID NO: 18, and a HCDR3 of SEQ ID NO: 19; and the second light chain variable region comprises a LCDR1 of SEQ ID NO: 20, a LCDR2 of SEQ ID NO: 21 and a LCDR3 of SEQ ID NO: 22.

In some embodiments, the second heavy chain variable region comprises or consists of an amino acid sequence of SEQ ID NO: 25 or an amino acid sequence having 95%, 96%, 97%, 98% or 99% sequence identity to the amino acid sequence of SEQ ID NO: 25 (with unchanged or substantially unchanged CDRs); and/or the second light chain variable region comprises or consists of an amino acid sequence of SEQ ID NO: 26 or an amino acid sequence having 95%, 96%, 97%, 98% or 99% sequence identity to the amino acid sequence of SEQ ID NO: 26 (with unchanged or substantially unchanged CDRs).

In some embodiments, the second heavy chain variable region has an amino acid sequence of SEQ ID NO: 25; and the second light chain variable region has an amino acid sequence of SEQ ID NO: 26.

In some embodiments, the bispecific antibody is a homodimer or heterodimer.

In some embodiments, the bispecific antibody is formed by fusion of antigen-binding fragments of D1 and D2, and has two mutually symmetric pairs of peptide chains, with each pair of peptide chains linked by a disulfide bond, wherein either pair of peptide chains has structures of formulas a to d from an N-terminus to a C-terminus:

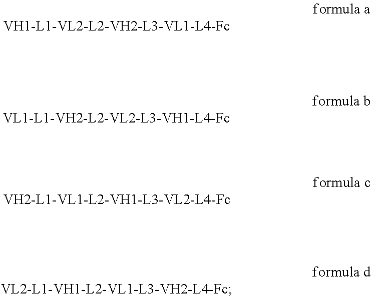

Wherein VH1 is the first heavy chain variable region, and VL1 is the first light chain variable region; VH2 is the second heavy chain variable region, and VL2 is the second light chain variable region; L1, L2, L3 and L4 are each independently none, a bond or a linker; Fc is an Fc element; and “-” represents a peptide bond.

In some embodiments, the Fc element comprises an Fc wild type or an Fc mutant type.

In some embodiments, the Fc element is derived from IgG1 or IgG4.

In some embodiments, the Fc mutant type is derived from an Fc fragment of IgG1.

In some embodiments, the Fc element has L234A and L235A mutations, and/or M252Y, S254T and T256E mutations.

In some embodiments, the linker is a rigid linker or a flexible linker.

In some embodiments, L1, L2, L3, L4 are each independently none or (GS)I, or (G4S)I (with I selected from 1 to 6).

In some embodiments, the VH1 comprises a HCDR1 of SEQ ID NO: 11, a HCDR2 of SEQ ID NO: 12 and HCDR3 of SEQ ID NO: 13, and the VL1 comprises a LCDR1 of SEQ ID NO: 14, a LCDR2 of SEQ ID NO: 15, and a LCDR3 of SEQ ID NO: 16.

In some embodiments, the VH2 comprises a HCDR1 of SEQ ID NO: 17, a HCDR2 of SEQ ID NO: 18, and a HCDR3 of SEQ ID NO: 19; and the VL2 comprises the second light chain variable region comprises a LCDR1 of SEQ ID NO: 20, a LCDR2 of SEQ ID NO: 21 and a LCDR3 of SEQ ID NO: 22.

In some embodiments, the bispecific antibody has an amino acid sequence of any one of SEQ ID NOs: 1 to 4 and 27.

In some embodiments, the bispecific antibody further comprises active fragments and/or derivatives of the bispecific antibody, and the derivatives of the bispecific antibody have at least 86%, 87%, 88%, 89%, 90%, 91%, 92%, 93%, 94%, 95%, 96%, 97%, 98%, or 99% sequence identity to the bispecific antibody of the present disclosure.

In a second aspect, the present disclosure provides a polynucleotide encoding the bispecific antibody as described in the first aspect of the present disclosure.

In a third aspect, the present disclosure provides a vector comprising the polynucleotide as described in the second aspect of the present disclosure.

In some embodiments, the vector comprises a plasmid, a phage, a yeast plasmid, a plant cell virus, a mammalian cell virus such as an adenovirus, a retrovirus, or other vector.

In a fourth aspect, the present disclosure provides a host cell, comprising the vector as described in the third aspect of the present disclosure or having a genome incorporating the polynucleotide as described in the second aspect of the present disclosure.

In some embodiments, the host cell is a eukaryotic cell, preferably a mammalian cell.

In a fifth aspect, the present disclosure provides a method for preparing the bispecific antibody as described in the first aspect of the present disclosure, comprising the steps of: (i) culturing the host cell described in the fourth aspect of the present disclosure under suitable conditions, to obtain a mixture comprising the bispecific antibody as described in the first aspect of the present disclosure; and (ii) purifying and/or separating the mixture obtained in step (i) to obtain the bispecific antibody as described in the first aspect of the present disclosure.

In a sixth aspect, the present disclosure provides a pharmaceutical composition, comprising: (I) the bispecific antibody as described in the first aspect of the present disclosure; and (II) a pharmaceutically acceptable carrier.

In some embodiments, the pharmaceutical composition further comprises a further pharmaceutically active reagent.

In some embodiments, the further pharmaceutically active reagent comprises a further antiviral reagent, for example, favipiravir, remdesivir or interferon. In some embodiments, the pharmaceutical composition is in a form of nasal spray, oral preparation, suppository or parenteral preparation.

In some embodiments, the nasal spray is selected from an aerosol, a spray and a powder inhaler.

In some embodiments, the oral preparation is selected from tablets, powders, pills, pulvis, granules, fine granules, soft/hard capsules, film-coated agents, pilules, sublingual tablets and ointment.

In some embodiments, the parenteral preparation comprises a transdermal agent, ointment, plaster, a topical liquid, or an injectable or pushable preparation.

In a seventh aspect, the present disclosure provides an immunoconjugate, comprising: (a) the bispecific antibody as described in the first aspect of the present disclosure; and (b) a conjugated moiety selected from the group consisting of: a detectable marker, a drug, a toxin, a cytokine, a radionuclide, an enzyme, or combinations thereof.

In some embodiments, the conjugated moiety is partially selected from: a fluorescent or luminescent marker, a radioactive marker, an MRI (Magnetic Resonance Imaging) or CT (Computerized Tomography) contrast agent, or an enzyme that is capable of producing a detectable product, a radionuclide, a biotoxin, a cytokine (e.g. IL-2, etc.), an antibody, an antibody Fc fragment, an antibody scFv fragment, a gold nanoparticle/nanorod, a virus particle, a liposome, or a magnetic nanoparticle.

In an eighth aspect, the present disclosure provides use of the bispecific antibody as described in the first aspect of the present disclosure, the pharmaceutical composition as described in the sixth aspect of the present disclosure, or the immunoconjugate as described in the seventh aspect of the present disclosure, in the preparation of (a) a detection reagent or kit; and/or (b) a medicament for preventing and/or treating infections caused by novel coronaviruses.

In a ninth aspect, the present disclosure provides a kit, comprising the bispecific antibody as described in the first aspect of the present disclosure, the polynucleotide as described in the second aspect of the present disclosure, the vector as described in the third aspect of the present disclosure, the host cell as described in the fourth aspect of the present disclosure, the pharmaceutical composition as described in the sixth aspect of the present disclosure, or the immunoconjugate as described in the seventh aspect of the present disclosure.

In a tenth aspect, the present disclosure provides use of the bispecific antibody as described in the first aspect of the present disclosure, the polynucleotide as described in the second aspect of the present disclosure, the vector as described in the third aspect of the present disclosure, the host cell as described in the fourth aspect of the present disclosure, the pharmaceutical composition as described in the sixth aspect of the present disclosure, or the immunoconjugate as described in the seventh aspect of the present disclosure in the preparation of a medicament for prevention, treatment, and/or detection of infections caused by novel coronaviruses; and preferably, the novel coronaviruses are SARS-CoV-2 prototype strains and/or SARS-CoV-2 mutant strains.

In some embodiments, the SARS-CoV-2 variant strain is selected from: SARS-CoV-2 Alpha (B.1.1.7), Beta (B.1.351), Gamma (P.1), Kappa (B.1.617.1), Delta (B.1.617.2) variant strains and Omicron (B.1.1.529) and sub-variant strains thereof.

In some embodiments, the novel coronaviruses are SARS-CoV-2 selected from the group consisting of: prototype strains, and mutant strains BA.1, BA.1.1, BA.2, BA.2.12.1, BA.2.75, BA.3, BA.4, BA.5, BF.7, BQ.1, BQ.1.1, XBB, XBB.1.5, XBB.1.16, and EG.5.

In an eleventh aspect, the present disclosure provides a method for preventing, treating and/or testing infections caused by novel coronaviruses, comprising: administering to a subject in need thereof a safe and effective amount of the bispecific antibody as described in the first aspect of the present disclosure, the pharmaceutical composition as described in the sixth aspect of the present disclosure, or the immunoconjugate as described in the seventh aspect of the present disclosure.

It should be understood that each of the above technical features of the present disclosure and each of the technical features specifically described below (e.g., in the Examples) can be combined with each other within the scope of the present disclosure to thus form new or preferred technical solutions.

### BRIEF DESCRIPTION OF THE DRAWINGS

FIG. 1A shows a schematic structural diagram of a bispecific antibody in which the light and heavy chain variable regions of a CoV56 monoclonal antibody (referred to as 56) and an L4.65 monoclonal antibody (referred to as 65) are arranged in a different order, respectively;

FIG. 1B shows a protein structure of the bispecific antibody according to the present disclosure;

FIG. 1C shows binding epitopes of the monoclonal antibodies 65 and 56 for RBD regions of an S protein of 2019-nCov.

FIG. 2A shows the binding capacity of a novel coronavirus antibody to 2019-nCov as determined by the enzyme-linked immunosorbent assay;

FIG. 2B shows the binding capacity of the novel coronavirus antibody to BA.1.1 as determined by the enzyme-linked immunosorbent assay;

FIG. 2C shows the binding capacity of the novel coronavirus antibody to BA.2.12.1 as determined by the enzyme-linked immunosorbent assay;

FIG. 2D shows the binding capacity of the novel coronavirus antibody to BA.5 as determined by the enzyme-linked immunosorbent assay;

FIG. 2E shows the binding capacity of the novel coronavirus antibody to BF.7 as determined by the enzyme-linked immunosorbent assay;

FIG. 2F shows the binding capacity of the novel coronavirus antibody to BQ.1.1 as determined by the enzyme-linked immunosorbent assay;

FIG. 2G shows the binding capacity of the novel coronavirus antibody to XBB as determined by the enzyme-linked immunosorbent assay.

FIG. 3 shows the neutralizing activity of DIA-19 against the pseudo-viruses of different novel coronavirus mutant strains, with L4.65 denoting the monoclonal antibody 65, Cov56 denoting the monoclonal antibody 56, Cocktail denoting the combined administration of the monoclonal antibodies 65 and 56, and wild type (prototype) denoting 2019-nCov.

FIG. 4A shows the neutralizing capacity of the novel coronavirus antibody against a live virus 2019-nCov (PT);

FIG. 4B shows the neutralizing capacity of the novel coronavirus antibody against a live virus Delta;

FIG. 4C shows the neutralizing capacity of the novel coronavirus antibody against a live virus BA.5.2;

FIG. 4D shows the neutralizing capacity of the novel coronavirus antibody against a live virus BF.7;

FIG. 4E shows the neutralizing capacity of the novel coronavirus antibody against a live virus BQ.1;

FIG. 4F shows the neutralizing capacity of the novel coronavirus antibody against a live virus BQ.1.1;

FIG. 4G shows the neutralizing capacity of the novel coronavirus antibody against a live virus XBB.

FIG. 5 shows the neutralization synergy of DIA-19 against a SARS-CoV-2 EG.5 mutant pseudo-virus.

FIG. 6A shows the change in body weight of mice after infection with SARS-CoV-2 XBB strains in a DIA-19 prevention experiment;

FIG. 6B shows the viral loads in lungs and nasal cavities of mice after infection with the SARS-CoV-2 XBB strains in the DIA-19 prevention experiment, ns: P>0.05; *: p<0.05; **: p<0.01; ***: p<0.001; and ****: p<0.0001.

FIG. 7A shows the change in body weight of mice after infection with SARS-CoV-2 XBB strains in a DIA-19 intraperitoneal injection treatment experiment;

FIG. 7B shows the viral loads in lungs and nasal cavities of mice after infection with the SARS-CoV-2 XBB strains in the DIA-19 intraperitoneal injection treatment experiment, ns: P>0.05; *: p<0.05; **: p<0.01; ***: p<0.001; and ****: p<0.0001.

FIG. 8 shows the viral copy number (Log 10 copies/g) in lung and brain tissues of mice on Day 3 (3 dpi) after infection with SARS-CoV-2 Delta strains in a prevention experiment, ns: P>0.05; *: p<0.05; **: p<0.01; ***: p<0.001; and ****: p<0.0001.

FIG. 9 shows the viral copy number (Log 10 copies/g) in lung and brain tissues of mice on Day 3 (3 dpi) after infection with the SARS-CoV-2 Delta strains in a treatment experiments, ns: P>0.05; *: p<0.05; **: p<0.01; ***: p<0.001; and ****: p<0.0001.

### DETAILED DESCRIPTION

Based on extensive and in-depth research and extensive screening, the inventors have unexpectedly constructed a bispecific antibody targeting different epitopes of the RBD region of a novel coronavirus with high specificity, high affinity and high broad-spectrum capacity for the first time. Specifically, the novel bispecific antibody is engineered by the unique fusion of two strains of non-competitive ultrabroad-spectrum fully human monoclonal antibodies CoV56 and L4.65 isolated from subjects immunized with recombinant protein vaccines of novel coronaviruses, as disclosed in Chinese patent applications 202211588062.X and 202211538937.5. Compared with the parent monoclonal antibody, the novel bispecific antibody has higher inhibitory activity and broad-spectrum capacity against all the tested Omicron strains (including substrains BA.5.2, BF.7 and XBB that are currently the most prevalent in China). The present disclosure has been completed on this basis.

### Terms

Unless otherwise defined, all technical and scientific terms used herein have the same meaning as commonly understood by those of ordinary skill in the art to which the present disclosure belongs.

The term “about” may refer to a value or set that is within an acceptable error range for a particular value or set as determined by those of ordinary skill in the art, and the value or set partially depends on how the value or set is measured or determined.

As used herein, the terms “contain” or “comprise (comprising)” may be open-ended, semi-closed and closed-ended. In other words, these terms also include “consisting essentially of”, or “consisting of”.

Typically, an “antibody” is also referred to as an “immunoglobulin” that may be a natural or conventional antibody, in which two heavy chains are linked to each other by a disulfide bond and each heavy chain is linked to a light chain by a disulfide bond. There are two types of light chains, namely, λ(l) and λ(k). There are five main heavy chain types (or isotypes), which determine the functional activity of antibody molecules: IgM, IgD, IgG, IgA, and IgE. Each type of chain comprises a different sequence of domains. The light chain comprises two domains or regions, namely, a variable domain (VL) and a constant domain (CL). The heavy chain comprises four domains, namely, a heavy chain variable region (VH) and three constant regions (CH1, CH2 and CH3, collectively referred to as CH). The variable regions of both the light chain (VL) and the heavy chain (VH) determine the binding recognition and specificity for antigens. The light chain's constant domain (CL) and the heavy chain's constant regions (CHs) give important biological properties, such as antibody chain binding, secretion, transplacental mobility, complement binding and binding to an Fc receptor (FcR). An Fv fragment is an N-terminal moiety of an immunoglobulin Fab fragment and consists of the variable moieties of one light chain and one heavy chain. The specificity of an antibody depends on the structural complementarity between an antibody binding site and an antigen determining interval. The antibody binding site consists of residues originating primarily from highly variable regions or complementary determining regions (CDRs). Occasionally, residues originating from non-highly variable or framework regions (FR) affect the overall domain structure and thus the binding site. The complementary determining regions or CDRs refer to amino acid sequences that collectively define the binding affinity and the specificity of the natural Fv region of the natural immunoglobulin binding site. The light and heavy chains of the immunoglobulin each have three CDRs, which are also referred to as CDR1-L, CDR2-L, CDR3-L and CDR1-H, CDR2-H, CDR3-H. The conventional antibody-antigen binding site thus comprises six CDRs, including a collection of CDRs from each v region of the heavy and light chains.

In the amino acid sequence of a light or heavy chain variable region of a given antibody, the exact amino acid sequence boundary of each CDR can be determined using any or a combination of a number of well-known antibody CDR assignment systems, which comprise, for example: Chothia based on the three-dimensional structure of the antibody and the topology of the CDR loop, Kabat based on the sequence variability of the antibody (Kabat, E., et al., U.S. Department of Health and Human Services, Sequences of Proteins of Immunological Interest, (1983)), AbM (University of Bath), Contact (University College London), International Immuno Gene Ticsdatabase (IMGT), the EU numbering system, and Chothia definition based on the loop structural position.

It should be understood that the exact amino acid sequence boundaries of the CDRs in the present disclosure may be optionally defined using the different assignment systems as mentioned above. Preferably, unless otherwise indicated, the positions of residues (including residues in both heavy and light chain variable regions) in the variable region of an antibody, when mentioned in the present disclosure, refer to positions numbered according to the Kabat numbering system.

As used herein, the term “variable” denotes that some moieties of the variable region of an antibody are somewhat different in sequence, which contribute to the binding and specificity of various particular antibodies to their particular antigens. However, variability is not uniformly distributed across the variable region of an antibody. It is concentrated in three fragments in the so-called complementary determining regions (CDRs) or hypervariable regions in the light and heavy chain variable regions. A conserved moiety in the variable region is called the framework region (FR). The variable regions of the natural heavy and light chains each comprise four FRs, which are roughly in a β-sheet configuration and linked by three CDRs that form a linking loop, and which in some cases may form a partially β-sheet structure. The CDRs in each chain are close together through the FRs and form, together with the CDRs of the other chain, the antigen-binding site of the antibody (see Kabat et al, NIH Publ. No. 91-3242, Vol. I, pp. 647-669 (1991)). The constant regions are not directly involved in antibody-antigen binding, but exhibit different effector functions. For example, they are involved the antibody-dependent cytotoxicity of the antibody.

As used herein, the term “framework region” (FR) refers to an amino acid sequence inserted between CDRs, that is, those moieties of the light and heavy chain variable regions of relatively conserved immunoglobulins among different immunoglobulins in a single species. The light and heavy chains of an immunoglobulin each have four FRs, referred to as FR1-L, FR2-L, FR3-L, FR4-L and FR1-H, FR2-H, FR3-H, FR4-H, respectively. Accordingly, the light chain variable domain may thus be referred to as (FR1-L)-(CDR1-L)-(FR2-L)-(CDR2-L)-(FR3-L)-(CDR3-L)-(FR4-L), and the heavy chain variable domain may thus be denoted as (FR1-H)-(CDR1-H)-(FR2-H)-(CDR2-H)-(FR3-H)-(CDR3-H)-(FR4-H). Preferably, the FR in the present disclosure is a human antibody FR or a derivative thereof, and the derivative of the human antibody FR is substantially identical to the FR of a naturally occurring human antibody, i.e., the sequence identity being up to 85%, 90%, 95%, 96%, 97%, 98% or 99%.

Once the amino acid sequence of the CDR is known, those skilled in the art can readily determine the framework regions FR1-L, FR2-L, FR3-L, FR4-L and/or FR1-H, FR2-H, FR3-H, FR4-H.

As used herein, the term “human framework region” is a framework region that is substantially identical (about 85% or more, specifically 90%, 95%, 97%, 99%, or 100%) to the framework region of a naturally occurring human antibody.

As used herein, the term “monoclonal antibody” or “mAb” refers to an antibody molecule having a single amino acid composition directed against a particular antigen, and it should not be construed that this antibody needs to be produced by any particular method. The monoclonal antibody may be produced from a single clone of a B cell or hybridoma, or may be recombinant, i.e., produced by protein engineering.

As used herein, the term “antigen” or “target antigen” refers to a molecule, or a moiety thereof, capable of being bound by an antibody or antibody-like binding protein. This term further refers to a molecule, or a moiety thereof, that can be used in an animal to produce an antibody capable of binding to an epitope of the antigen. The target antigen may have one or more epitopes. For each target antigen recognized by an antibody or an antibody-like binding protein, the antibody-like binding protein is capable of competing with an intact antibody that recognizes the target antigen.

As used herein, the term “affinity” is in theory defined by the equilibrium association between the intact antibody and the antigen. The affinity of the bispecific antibody of the present disclosure can be evaluated or determined with a KD value (a dissociation constant) (or by other assays), for example, by Bio-layer interferometry (BLI), using a Fortebio Red 96 instrument.

As used herein, the term “linker” refers to one or more amino acid residues that are inserted into the immunoglobulin structural domains to provide sufficient mobility to the light and heavy chain domains for pleating of the domains into an exchanged dual variable region immunoglobulin.

Examples of appropriate linkers include single glycine (Gly) or serine (Ser) residues, and the identifications and sequences of amino acid residues in the linker can vary depending on the type of secondary structure elements to be achieved in the linker. Preferred linkers may be (GS)I or (G4S)I (I selected from 1 to 6).

“ECMO” means extracorporeal membrane oxygenation (ECMO), which is a medical emergency technical device primarily used to provide continuous extracorporeal respiration and circulation to a patient with severe cardiorespiratory failure in order to maintain the patient's life.

“ICU” refers to an intensive care unit, in which treatment, care and rehabilitation can be carried out synchronously to provide a premise and devices for critically ill or comatose patients for combination of optimal care, comprehensive treatment and medical care, as well as for provision of early postoperative rehabilitation, joint care exercise therapy and other services.

“IMV” means intermittent mandatory ventilation, which is intended for implementation of cyclic volume or pressure ventilation based on a pre-set time interval, i.e. a time-triggered process. This allows a patient to breathe spontaneously at any set base pressure level during the mandatory ventilation. During spontaneous breathing, the patient can breathe spontaneously with the support of continuous airflow, or is allowed to breathe spontaneously by opening the valve by a machine on demand. Most ventilators can provide pressure support during spontaneous breathing.

“ECMO” means extracorporeal membrane oxygenation (ECMO), which is a medical emergency technical device primarily used to provide continuous extracorporeal respiration and circulation to a patient with severe cardiorespiratory failure in order to maintain the patient's life. At present, it can be applied to patients with acute hypoxemic respiratory failure, patients subjected to surgical procedures, patients with respiratory failure but without trachea cannula, immunocompromised patients, patients with heart failure, etc.

“NIV” means non-invasive ventilation, which is mechanical ventilation that is non-invasive except for trachea cannula or tracheostomy.

### Novel Coronavirus (SARS-CoV-2)

As used in herein, the terms “novel coronavirus”, “2019-nCoV”, or “SARS-CoV-2” can be used interchangeably. The 2019 novel coronavirus is the 7th coronavirus known to infect humans and leads to COVID-19, and it is one of the serious infectious diseases threatening the human health across the world.

Coronavirus (CoV) belongs to the family Coronaviridae of the order Nidovirales. It is an enveloped positive-stranded RNA virus, with subfamilies including four genera: α, β, δ and γ. Among the known coronaviruses that infect humans, HCoV-229E and HCoV-NL63 are coronaviruses of the alpha genus, while HCoV-OC43, SARS-CoV, HCoV-HKU1, MERS-CoV, and SARS-CoV-2 are coronaviruses of the beta genus. SARS-CoV-2 is also known as 2019-nCoV.

This type of virus has a genome of a single positive-stranded RNA, making it one of the largest RNA viruses in terms of genome. It encodes replicases, spike proteins, membrane proteins, envelope proteins, nucleocapsid proteins, etc. At the initial stage of viral replication, the genome is translated into two peptide chains, known as precursor polyproteins, of several thousand amino acids long. These precursor polyproteins are then cleaved by proteases to produce non-structural proteins (such as RNA polymerases and helicases) and structural proteins (such as spike proteins), as well as accessory proteins.

### Bispecific Antibody

As used herein, the terms “bispecific antibody of the present disclosure”, “bifunctional fusion antibody of the present disclosure”, and “bispecific antibody against coronavirus RBD of the present disclosure” can be used interchangeably, referring to a bispecific antibody that binds to both the first and second epitopes targeting the SARS-CoV-2 RBD domain.

In the present disclosure, the bispecific antibody comprises: a first targeting domain D1, which targets a first epitope of a SARS-CoV-2 RBD domain; and a second targeting domain D2, which targets a second epitope of the SARS-CoV-2 RBD domain; wherein the first epitope of the SARS-CoV-2 RBD domain has one or more (preferably 1, 2, 3, 4, 5, 6, 7, 8, 9, 10, 11, 12, 13, 14, 15, 16, 17 or 18) residues selected from the following positions in an RBD region of an S protein: 346R, 437N, 438S, 439N, 440N, 441L, 443S, 444K, 445V, 446G, 447G, 448N, 449Y, 450N, 498Q, 499P, 500T, and 506Q; and the second epitope of the SARS-CoV-2 RBD domain has one or more residues (preferably 1, 2, 3, 4, 5, 6, 7, 8, 9, 10, 11, 12, 13, 14, 15, 16 or 17) selected from the following positions in the RBD region of the S protein: 352A, 353W, 355R, 357R, 393T, 394N, 396Y, 462K, 463P, 464F, 465E, 466R, 468I, 516E, 518L, 519H, and 520A.

In some embodiments, the first epitope and the second epitope do not overlap or substantially do not overlap.

In a preferred embodiment, the bispecific antibody of the present disclosure is a homodimer or a heterodimer; preferably a homodimer having the structure of any of formulae a to d from the N-terminus to the C-terminus:

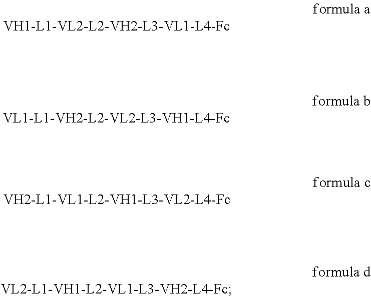

Wherein VH1 is the first heavy chain variable region, and VL1 is the first light chain variable region; VH2 is the second heavy chain variable region, and VL2 is the second light chain variable region; L1, L2, L3 and L4 are each independently none, a bond or a linker; Fc is an Fc element; and “-” represents a peptide bond.

The bispecific antibody of the present disclosure comprises not only an intact antibody, but also a fragment of an immunologically active antibody or a fusion protein formed by the antibody and other sequences. Hence, the present disclosure further comprises the fragment, derivative and analog of the antibody as described.

As used herein, the terms “fragment”, “derivative” and “analog” each refer to a polypeptide that maintains substantially the same biological function or activity as the antibody of the present disclosure. The polypeptide fragment, derivative or analog of the present disclosure may be: (i) a polypeptide having one or more conserved or non-conserved amino acid residues (preferably conserved amino acid residues) substituted in such a way that the substituted amino acid residues may or may not be encoded by the genetic code, or (ii) a polypeptide having substituent groups in one or more amino acid residues, or (iii) a polypeptide formed by fusion of a mature polypeptide with a further compound (e.g., a compound for extending the half-life period of the polypeptide, for example, polyethylene glycol); or (iv) a polypeptide formed by fusion of an additional amino acid sequence to the current polypeptide sequence (e.g., a leading sequence or a secretory sequence or a sequence for purifying the current polypeptide or a proteinogen sequence, or a fusion protein formed with a 6His tag). According to the teachings herein, these fragments, derivatives and analogs shall fall within the scope of what is known to those skilled in the art.

The antigen-binding fragment of the present disclosure comprises those capable of specifically binding to the coronavirus RBD. Examples of the antibody-binding fragment include, for example, but is not limited to, Fab, Fab′, F(ab′)2, a Fv fragment, a single-chain Fv (scFv) fragment, and a single-domain fragment.

The “Fab” fragment consists of one light chain and the CH1 and variable regions of one heavy chain.

The “Fab′” fragment comprises one light chain and the moiety of one heavy chain consisting of a VH domain, a CH1 domain, and a portion of the constant region between the CH1 and CH2 domains, and an interchain disulfide bond is formed between the two heavy chains of two Fab′ fragments to form a F(ab′)2 molecule.

The “F(ab′)2” fragment comprises two light chains and the moieties of two heavy chains consisting of VH domains, CH1 domains, and the portions of the constant regions between the CH1 and CH2 domains, whereby an interchain disulfide bond is formed between the two heavy chains. Hence, the F(ab′)2 fragment consists of two Fab′ fragments that are held together by an interchain disulfide bond between the two heavy chains.

The “Fv” fragment is the smallest fragment of the antibody comprising the complete target recognition and binding site. This region consists of a dimer (VH-VL dimer) of variable domains of one heavy chain and one light chain that are bound in a tight non-covalent manner. In this configuration, three CDRs in each variable domain interact to define the target binding site on the surface of the VH-VL dimer. Typically, six CDRs endow the antibody with the target binding specificity. In some cases, however, even a single variable domain (or only half of an Fv comprising only three CDRs with target specificity) may be able to recognize and bind a target, despite its lower affinity than the entire binding site.

The antibody-binding fragment “single-chain Fv” or “scFv” comprises the VH and VL domains of the antibody, with these domains present in a single polypeptide chain. Generally, the Fv polypeptide further comprises a polypeptide linker between the VH and VL domains, which enables the scFv to form a structure facilitating target binding.

The “single-domain fragment” consists of a single VH or VL domain that shows sufficient affinity to the coronavirus RBD. In a specific embodiment, the single-domain fragment is camelidized.

The bispecific antibody against coronavirus RBD of the present disclosure comprises a derivatized antibody. For example, the derivatized antibody is typically modified by glycosylation, acetylation, pegylation, phosphorylation, amidation, derivatization by known protecting/blocking groups, proteolytic cleavage, and attachment to a cellular ligand or other proteins. Any of numerous chemical modifications can be performed by known techniques, including but not limited to, specific chemical cleavage, acetylation, formylation, metabolic synthesis of tunicamycin, etc. In addition, the derivative may comprise one or more non-natural amino acids. For example, the ambrx technology is used.

The bispecific antibody of the present disclosure refers to the antibody that has anti-SARS-CoV-2 activity and comprises two chains each with a structure of any of the above formulae a to d. This term also comprises the variant forms of the antibody that has the same function as the bispecific antibody of the present disclosure and comprises two chains each with a structure of any of the above formulae a to d. These variant forms include (but are not limited to): the deletion, insertion and/or substitution of one or more (typically 1 to 50, preferably 1 to 30, more preferably 1 to 20, optimally 1 to 10) amino acids, and the addition of one or more (typically up to 20, preferably up to 10, more preferably up to 5) amino acids at the C-terminus and/or N-terminus. For example, the substitution with an amino acid having similar or analogous properties typically does not alter the function of the protein in the art. For another example, the addition of one or several amino acids to the C-terminus and/or the N-terminus also typically does not alter the function of the protein. This term also comprises active fragments and active derivatives of the bispecific antibody of the present disclosure.

Variant forms of the bispecific antibody include: homologous sequences, conserved variants, allelic variants, natural mutants, induced mutants, proteins encoded by DNA capable of hybridizing with the DNA encoding the antibody of the present disclosure under conditions of high or low stringency, and polypeptides or proteins obtained using antisera against the antibody of the present disclosure.

In the present disclosure, the “conserved variants of the bispecific antibody of the present disclosure” refer to polypeptides formed by substitution of up to 10, preferably up to 8, preferably up to 5, and optimally up to 3 amino acids with amino acids having similar or analogous properties, as compared to the amino acid sequence of the bispecific antibody of the present disclosure. These conserved variant polypeptides are preferably produced by amino acid substitution according to Table 1.

TABLE 1

| | | Preferred |
|---|---|---|
| Initial residues | Representative substitution | substitution |
| Ala(A) | Val; Leu; Ile | Val |
| Arg(R) | Lys; Gln; Asn | Lys |
| Asn(N) | Gln; His; Lys; Arg | Gln |
| Asp(D) | Glu | Glu |
| Cys(C) | Ser | Ser |
| Gln(Q) | Asn | Asn |
| Glu(E) | Asp | Asp |
| Gly(G) | Pro; Ala | Ala |
| His(H) | Asn; Gln; Lys; Arg | Arg |
| Ile(I) | Leu; Val; Met; Ala; Phe | Leu |
| Leu(L) | Ile; Val; Met; Ala; Phe | Ile |
| Lys(K) | Arg; Gln; Asn | Arg |
| Met(M) | Leu; Phe; Ile | Leu |
| Phe(F) | Leu; Val; Ile; Ala; Tyr | Leu |
| Pro(P) | Ala | Ala |
| Ser(S) | Thr | Thr |
| Thr(T) | Ser | Ser |
| Trp(W) | Tyr; Phe | Tyr |
| Tyr(Y) | Trp; Phe; Thr; Ser | Phe |
| Val(V) | Ile; Leu; Met; Phe; Ala | Leu |

### Encoding Nucleic Acid and Expression Vector

The present disclosure further provides a polynucleotide molecule encoding the antibodies or fragments thereof or fusion proteins thereof as described above. The polynucleotide of the present disclosure may be in a DNA or RNA form. The DNA form includes cDNA, genomic DNA or synthetic DNA. The DNA may be single-stranded or double-stranded. The DNA may be a coding or non-coding strand.

The polynucleotide encoding a mature polypeptide of the present disclosure includes: a coding sequence encoding only the mature polypeptide; a coding sequence of the mature polypeptide and various additional coding sequence; a coding sequence (and optionally additional coding sequences) and a non-coding sequence of the mature polypeptide.

The term “polynucleotide encoding a polypeptide” may be a polynucleotide comprising the encoded polypeptide, or a polynucleotide comprising an additional coding and/or non-coding sequence(s).

The nucleic acid (and combination of nucleic acids) of the present disclosure may be used to produce a recombinant antibody of the present disclosure in a suitable expression system.

The present disclosure further relates to a polynucleotide that hybridizes with the sequences described above and has at least 50%, preferably at least 70%, or more preferably at least 80% identity between the two sequences. The present disclosure relates in particular to a polynucleotide which is hybridizable with the polynucleotide described in the present disclosure under stringent conditions. In the present disclosure, the “stringent conditions” refer to: (1) hybridization and elution at lower ionic strength and higher temperature, such as 0.2×SSC, 0.1% SDS, 60° C.; or (2) hybridization with a denaturant added, such as 50% (v/v) formamide, 0.1% calf serum/0.1% Ficoll, 42° C., etc.; or (3) hybridization occurring only when the identity between the two sequences is at least 90%, preferably 95%. Moreover, the polypeptide encoded by the hybridizable polynucleotide has the same biological function and activity as the mature polypeptide.

The full-length nucleotide sequence of the antibody of the present disclosure or fragments thereof can typically be obtained by PCR amplification, recombination or synthetic methods. A possible method is to synthesize a related sequence by the synthetic method, especially when the fragment length is short. Typically, a fragment with a very long sequence can be obtained by first synthesizing a plurality of small fragments and then ligating them. In addition, the heavy chain coding sequences and expression tags (e.g., 6His) can be fused together to form a fusion protein.

Once the related sequence has been obtained, the recombination method may be used to obtain the sequence on a large scale. This is typically done by cloning into a vector, transferring into a cell, and then isolating the related sequence from proliferated host cells by a conventional method. The biomolecules (nucleic acids, proteins, etc.) involved in the present disclosure comprise biomolecules in isolated form.

At present, it is already possible to obtain a DNA sequence encoding a protein of the present disclosure (or a fragment thereof, or a derivative thereof) exclusively by chemical synthesis. Then, this DNA sequence can be introduced into various existing DNA molecules (or, for example, vectors) and cells known in the art. In addition, mutations may be introduced into the protein sequence of the present disclosure by chemical synthesis.

The present disclosure further relates to vectors comprising appropriate DNA sequences as described above, as well as appropriate promoters or control sequences. These vectors can be used to transform appropriate host cells to enable them to express the protein.

The host cells may be prokaryotic cells, such as bacterial cells; or lower eukaryotic cells, such as yeast cells; or higher eukaryotic cells, such as mammalian cells. Representative examples include: bacterial cells of Escherichia coli, Streptomyces spp. and Salmonella typhimurium; fungal cells such as yeast; insect cells of Drosophila S2 or Sf9; and animal cells such as CHO, COS7 and 293 cells, etc.

Transformation of the host cells with recombinant DNAs can be performed by conventional techniques known to those skilled in the art. When the host is a prokaryote such as Escherichia coli, competent cells capable of absorbing DNA can be harvested after an exponential growth period and then treated with the CaCl2 method, with the steps well known in the art. Another method includes the use of MgCl2. The transformation may also be performed by electroporation, if desired. When the host is an eucaryon, the following DNA transfection methods may be used: calcium phosphate co-precipitation, conventional mechanical methods such as microinjection, electroporation, liposome packaging, etc.

The obtained transformants may be cultured by conventional methods to express the polypeptide encoded by the genes of the present disclosure. Depending on the host cells used, a medium used during culturing may be selected from a variety of conventional media. The culture is carried out under conditions suitable for the growth of the host cells. After the host cells have grown to an appropriate cell density, a selected promoter is induced with an appropriate method (e.g., temperature shift or chemical induction), and the cells are cultured for an additional period of time.

The recombinant polypeptides in the methods above may be expressed inside the cells or on the cell membranes, or secreted outside the cells. The recombinant proteins may be isolated and purified by various separation methods using their physical, chemical and other properties, if desired. These methods are well known to those skilled in the art. Examples of these methods include but are not limited to: conventional renaturation, treatment with a protein precipitant (a salting-out method), centrifugation, osmotic lysis, ultra-treatment, ultra-centrifugation, molecular sieve chromatography (gel filtration), adsorption chromatography, ion exchange chromatography, high-performance liquid chromatography (HPLC), and various other liquid chromatography techniques, and combinations of these methods.

The bispecific antibody of the present disclosure may be used alone or in combination, binding or conjugation with detectable markers (for a diagnostic purpose), therapeutic agents, or any combination thereof.

The detectable markers for the diagnostic purpose include, but are not limited to: a fluorescent or luminescent marker, a radioactive marker, an MRI (Magnetic Resonance Imaging) or CT (Computerized Tomography) contrast agent, or an enzyme capable of producing a detectable product.

The therapeutic agents that can be bound or conjugated to the antibody of the present disclosure include, but are not limited to: 1, a radionuclide; 2, a biotoxin; 3, a cytokine such as IL-2, etc.; 4, a gold nanoparticle/nanorod; 5, a viral particle; 6, a liposome; 7, a magnetic nanoparticle; and 8, a medicament intended for neutralizing a virus, including but not limited to, favipiravir, remdesivir, or interferon, etc.

### Pharmaceutical Composition

The administration dose of the active ingredient in the pharmaceutical composition of the present disclosure varies depending on the subject of administration, the subject's organ, symptoms, the method of administration, etc., and may be determined based on the judgment of a physician by considering the type of dosage form, the method of administration, the age and body weight of the patient, and the symptoms of the patient, etc.

The pharmaceutical composition of the present disclosure comprises the bispecific antibody, or an active fragment thereof or a fusion protein thereof, in the present disclosure as described above, and a pharmaceutically acceptable carrier. Typically, these substances may be formulated in a non-toxic, inert, and pharmaceutically acceptable aqueous carrier medium, with the pH typically of about 5-8, and preferably about 6-8, although the pH may vary depending on the nature of the substance to be formulated and the condition to be treated. The formulated pharmaceutical composition may be administered by conventional routes, including (but not limited to): intravenous injection, intravenous drip, subcutaneous injection, topical injection, intramuscular injection, intratumoral injection, intraperitoneal injection (e.g., inside peritoneum), intracranial injection, intracavity injection, or intranasal administration (e.g., nasal spray administration). In particular, the pharmaceutical composition described herein may be formulated for intranasal administration or administration by another topical route, for example, administration to a biological surface, including for example a mucous membrane or skin. Drug carriers suitable for facilitating this mode of administration are well known in the art.

Preferably, the pharmaceutical composition of the present disclosure further comprise other pharmaceutically active agents, for example, other antiviral agents, preferably favipiravir, remdesivir or interferons.

Specifically, a nasal spray containing 0.001% or 0.15% (w/w) azelastine compound in an aqueous solution at pH 6.8±0.3 may be used, which optionally further contains any one or more hydroxypropyl methylcellulose in citric acid monohydrate, disodium hydrogen phosphate dodecahydrate or disodium edetate, purified water, sodium chloride, benzalkonium chloride and other preservatives.

As used herein, in terms of the treatment of a subject or the administration or application of a preparation of a corresponding formulation or other mucosal use, the term “mucosal” refers to administration by the mucosal route, including systemic or topical administration, in which an active ingredient is absorbed by contact with a mucosal surface. This encompasses nasal, pulmonary, oral or buccal administration and formulations, such as liquids, syrups, lozenges, eye drops, tablets, sprays, powders, instant powders, granules, capsules, creams, gels, drops, suspensions or emulsions.

The bispecific antibody or the antigen-binding fragment thereof of the present disclosure or the pharmaceutical composition of the present disclosure can be administered to a subject by any appropriate administration routes. Such routes include, but are not limited to, oral, buccal, sublingual, topical, parenteral, rectal, intrathecal or nasal routes.

As used herein, the term “parenteral” refers to modes of administration that include intravascular, intramuscular, intranasal, intraperitoneal, intrathoracic, subcutaneous, and intraarticular injections and infusions. The mode of administration may be systemic or topical.

As used herein, the term “safe and effective amount” may vary depending on the subject of administration, the subject's organ, symptoms, and the method of administration, etc., and may be determined based on the judgement of a physician by considering the type of dosage form, the method of administration, the age and body weight of the patient, the symptoms of the patient, etc.

The pharmaceutical composition of the present disclosure comprises a safe and effective amount (e.g., 0.001 wt % to 99 wt %, preferably 0.01 wt % to 90 wt %, more preferably 0.1 wt % to 80 wt %) of the bispecific antibody (or its conjugate) of the present disclosure as described above, and a pharmaceutically acceptable carrier or excipient. Such carriers include (but are not limited to): saline, buffer solutions, glucose, water, glycerol, ethanol, and their combination. The pharmaceutical preparation should match the route of administration. The pharmaceutical composition of the present disclosure may be prepared into an injectable form, for example, by conventional methods using normal saline or aqueous solutions containing glucose and other excipients. The pharmaceutical composition in a form such as an injection or a solution should be manufactured under sterile conditions. The administration dose of an active ingredient is a therapeutically effective amount, for example, about 10 micrograms per kilogram of body weight to about 50 micrograms per kilogram of body weight on a daily basis. In addition, the polypeptide of the present disclosure may also be used in conjunction with other therapeutic agents.

When the pharmaceutical composition is used, a safe and effective amount of the immunoconjugate is administered to a mammal, with the safe and effective amount typically of at least about 10 micrograms per kilogram of body weight, and in most cases not more than about 50 milligrams per kilogram of body weight, preferably about 10 micrograms to about 10 milligrams per kilogram of body weight. Without doubt, the specific dose should also take factors such as the route of administration and the patient's health status into consideration, all of which fall within the area of expertise of skilled physicians.

### Combined Therapy

In some embodiments, the bispecific antibody of the present disclosure may be combined with other therapeutic or preventive protocols, including the administration of one or more antiviral antibodies and one or more further therapeutic agents or methods together or in combination. For the combined therapy, the bispecific antibody of the present disclosure may be administered with other therapeutic agents simultaneously or separately. In the case of separate administration, the bispecific antibody of the present disclosure may be administered before or after the administration of a further therapeutic agent.

In some embodiments, the therapeutic agents used in combination with the bispecific antibody of the present disclosure include one of the following: HIV drugs, antimalarial drugs, RNA polymerase inhibitors, antiviral drugs, and monoclonal-antibody drugs. In some embodiments, the HIV drugs include Lopinavir/ritonavir, ASC09/ritonavir, and darunavir; and the exclusive use of lopinavir/ritonavir and ribavirin is not recommended. In some embodiments, the antimalarial drugs include chloroquine phosphate. In some embodiments, the antiviral drugs include abidol, favipiravir, and α-interferons. In some embodiments, the monoclonal-antibody drugs include BDB-001.

In some patients with severe or critical infections of the novel coronaviruses, cytokine storm occurs, which can be treated by the combination of the antibody of the present disclosure and adalimumab (and for example its biosimilars, such as Abrilada™ (adalimumab-afzb), Amjevita (adalimumab-att), Cyltezo™ (adalimumab-adbm), Hyrimoz™ (adalimumab-adaz), Hulio™ (BAT1406)) or tochilizumab (and for example its biosimilars, such as BAT1806), which can slow down the inflammatory response caused by the upregulated TNF-α expression. In some embodiments, the patients treated by the present method are diagnosed with a novel coronavirus infection and have one or more elevated cytokines (including tumor necrosis factor α (TNF-α), IFN-γ, IL-1β, IL-2, IL-4, IL-7, IL-8, IL-10, IL-12p70, IL-13, granulocyte colony-stimulating factor (GSCF), interferon-induced protein-10 (IP-10), monocyte chemotactic protein-1 (MCP1), and macrophage inflammatory protein 1α (MIP1A)). In some embodiments, the patients treated by the present method have increased TNF-α. In some embodiments, one or more cytokines are elevated by at least 50% above a normal level. In some embodiments, one or more cytokines are at least 2 times, 3 times, or 4 times the normal level. In some embodiments, before treatment by the present method, the patients have fever, hypotension, hypoxia, and/or acute respiratory distress syndrome (ARDS). In some embodiments, before treatment by the present method, the patients have lungs filled with inflammatory fluid (commonly referred to as “white lung”). In some embodiments, before treatment by the present method, the patients have cytokine release syndrome (CRS) caused by a cytokine storm.

In some embodiments, the bispecific antibody of the present disclosure is used in combination with ICU treatment. In some embodiments, the bispecific antibody of the present disclosure is combined with extracorporeal membrane oxygenation (ECMO) and/or invasive mechanical ventilation (IMV) treatment. In some embodiments, the bispecific antibody of the present disclosure is combined with the oxygen therapy. In some embodiments, the bispecific antibody of the present disclosure is combined with the NIV/HFNC therapy. In some embodiments, after treatment, one or more cytokines in the patient are reduced by at least 20%, 30%, 40%, 50%, 60%, 70%, 80%, 90% or 95% as compared with that before treatment. In some embodiments, the present method leads to the recovery of the patient.

### Application

The present disclosure further provides a diagnostic method and uses. In some embodiments, there is provided a method for detecting the expression of SARS-CoV-2 in a sample, comprising: contacting the sample with the antibody to allow the antibody to bind to a spike protein, and detecting the binding, i.e., the content of the spike protein in the sample. The present disclosure provides a bispecific antibody targeting coronaviruses and applications thereof, where a first targeting domain and a second targeting domain in the bispecific antibody synergistically prevent cell infection by SARS-CoV-2 virus particles, and mediate immune cell phagocytosis and clearance of virus particles, to prevent, treat, or ameliorate COVID-19; and the bispecific antibody of the present disclosure may also be used to diagnose whether a patient is infected with SARS-CoV-2. In some embodiments, there is provided an application of the antibody in preparation of a kit for diagnosis of COVID-19 or for the detection of SARS-CoV-2 antigens. In some embodiments, there is provided a diagnostic kit comprising the bispecific antibody as described.

The present disclosure further provides a treatment method and uses. In some embodiments, there is provided a method for preventing, treating, or ameliorating COVID-19, comprising: administering to a patient an effective dose of the bispecific antibody of the present disclosure. In some embodiments, there is provided an application of the bispecific antibody in prevention, treatment, or amelioration of COVID-19. In some embodiments, there is provided an application of the bispecific antibody in preparation of a medicament for preventing, treating, or ameliorating COVID-19. In some embodiments, the patient is a suspected case of SARS-CoV-2 virus infection. In some embodiments, the patient is the one who has been in contact with a SARS-CoV-2 virus carrier. In some embodiments, the patient is the one who has been diagnosed with infection by the SARS-CoV-2 virus. In some embodiments, the patient is the one with mild symptoms. In some embodiments, the patient is the one with severe symptoms. In some embodiments, the patient may have fever, cough, hypotension, hypoxia, and/or acute respiratory distress syndrome (ARDS).

The specific dose and therapeutic regimen for any particular patient will depend on various factors, including the antibodies used, the age and body weight of the patient, the general health status, gender and diet, as well as the timing of administration, frequency of excretion, drug combinations, and the severity of the particular being treated. These factors will be assessed by healthcare personnel within the scope of those ordinarily skilled in the art. The dose will also depend on the individual patient being treated, the route of administration, the type of formulation, the characteristics of a compound used, the severity of the disease, and the desired effects. The dose used may be determined by means of the pharmacological and pharmacokinetic principles known in the art.

The main advantages of the present disclosure are as follows.

(1) The bispecific antibody of the present disclosure has broad-spectrum neutralizing activity, showing good neutralizing capacity against the pseudo-viruses of currently prevalent novel coronavirus strains and better broad-spectrum and neutralizing capacity than those of the positive control LY-CoV1404, the parent monoclonal antibody, and the combined therapy of the parent monoclonal antibody (a cocktail therapy, i.e., the combined administration of monoclonal antibodies 65 and 56).

(2) The bispecific antibody of the present disclosure can effectively inhibit SARS-CoV-2 pseudo-virus infection, and exhibit good neutralizing activity against authentic viruses. The bispecific antibody of the present disclosure has application value in the clinical treatment and prevention of SARS-CoV-2 infection.

(3) The bispecific antibody of the present disclosure can effectively inhibit the replication of SARS-CoV-2 strains in the lungs, nasal cavities, and brains of transgenic mice, providing a good preventive, therapeutic and protective effect in mice. The present disclosure provides a potential novel antibody medicament for the clinical prevention, treatment, and detection of the novel coronaviruses.

The following further describes the present disclosure in conjunction with specific examples. It should be understood that these examples are intended only for illustrating the present disclosure, rather than limiting the scope of the present disclosure. Experimental methods not specified in detail in the following examples generally follow the standard conditions as described in Sambrook et al., Molecular Cloning: A Laboratory Manual (New York: Cold Spring Harbor Laboratory Press, 1989), or according to the conditions recommended by the manufacturer. Unless otherwise stated, percentages and parts are calculated by weight.

### Example 1. Construction of Bispecific Antibody Protein Molecules

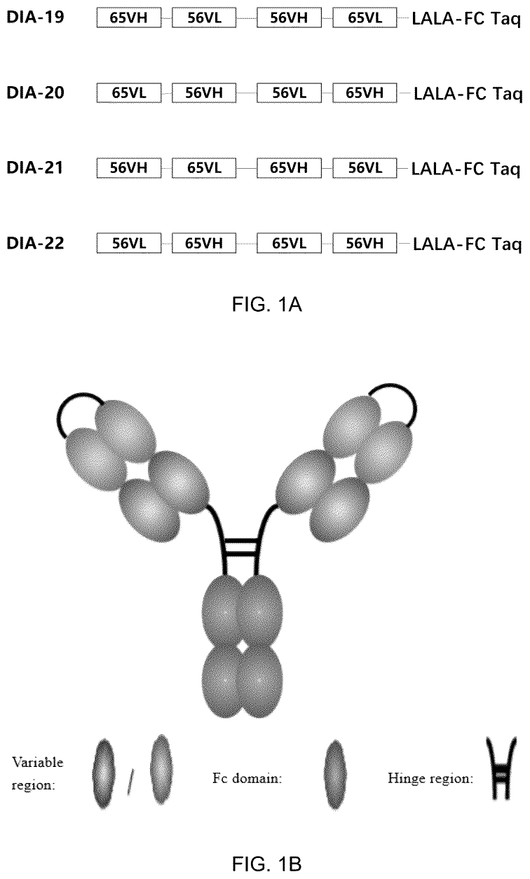

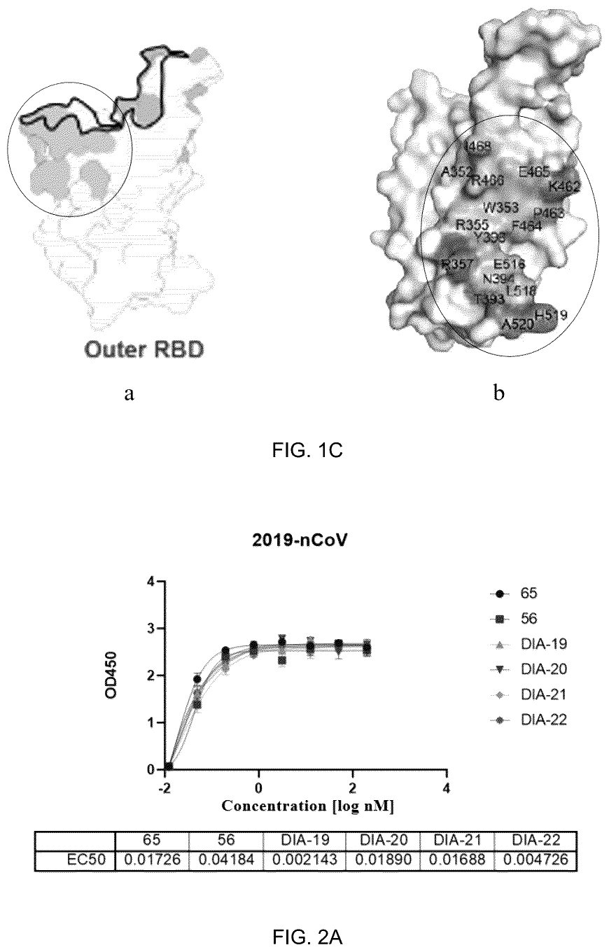

With two strains of non-competitive ultrabroad-spectrum fully human monoclonal antibodies CoV56 and L4.65 isolated from subjects immunized with recombinant protein vaccines of novel coronaviruses as disclosed in Chinese patent applications 202211588062.X and 202211538937.5, the variable regions of the light and heavy chains of the monoclonal antibodies CoV56 (referred to as the monoclonal antibody 56 or 56IgG) and L4.65 (referred to as the monoclonal antibody 65 or 65IgG) were arranged in different orders respectively, and after the introduction of mutations L234A and L235A, linked to FC taq (as shown in FIG. 1A), to construct bispecific antibody proteins DIA-19 (SEQ ID NO. 1), DIA-20 (SEQ ID NO. 2), DIA-21 (SEQ ID NO. 3) and DIA-22 (SEQ ID NO. 4), with the structures shown in FIG. 1B.

Furthermore, based on the structure of DIA-19, a bispecific antibody protein DIA-23 (SEQ ID NO. 27) with mutations M252Y/S254T/T256E in the Fc region was further constructed to extend the half-life of the bispecific antibody.

The seven-class definition method proposed by Hastie K M et al. (see Hastie K M et al: Defining variant-resistant epitopes targeted by SARS-CoV-2 antibodies: A global consortium study. Science 2021, 374(6566): 472-478) was used to determine the binding epitopes of the monoclonal antibodies 65 and 56 in the present disclosure (as shown in FIG. 1C).

Based on experiments, the binding epitope of the monoclonal antibody 65 was found in the RBD5 region of the S protein. The residues were as follows: 2019-nCoV: 346R, 437N, 438S, 439N, 440N, 441L, 443S, 444K, 445V, 446G, 447G, 448N, 449Y, 450N, 498Q, 499P, 500T and 506Q.

The crystal structure of a complex of the monoclonal antibody 56 with the SARS-CoV-2 RBD was analyzed to obtain the RBD epitope recognized by the monoclonal antibody 56. The binding epitope of the monoclonal antibody 56 was found in the RBD region of the S protein that is different from that of the binding epitope of the monoclonal antibody 65. The residues were as follows: 2019-nCoV: 352A, 353W, 355R, 357R, 393T, 394N, 396Y, 462K, 463P, 464F, 465E, 466R, 468I, 516E, 518L, 519H and 520A.

The binding epitopes of the monoclonal antibodies 65 and 56 were at non-overlapping positions within the RBD region, and the RBD region where the binding of the monoclonal antibody 56 occurs was relatively conserved.

### Example 2. Determination of the Binding Capacity of Bispecific Antibodies Against Novel Coronaviruses to RBDs of Various Novel Coronaviruses Strains by Enzyme-Linked Immunosorbent Assay (ELISA)

The RBD proteins (2019-nCoV, BA.1.1, BA.2.12.1, BA.5, XBB, BF.7, BQ.1.1) were diluted to 1 μg/mL with a buffer solution, added to the ELISA plates at 100 μL/well, and allowed to stand overnight at 4° C.; and the plates were washed with PBST three times, a blocking solution was added at 300 μL/well, and the plates were allowed to stand for 2 hours at room temperature and then washed with PBST three times for later use. The bispecific antibodies were diluted with a diluent by 4-fold dilution to form 8 concentration gradients, with the starting concentrations of 200 nM (BA.1.1, BA.2.12.1 and XBB), 100 nM (BQ.1.1), 50 nM (BF.7) or 30 nM (2019-nCoV and BA.5), respectively, and then these diluted bispecific antibodies were sequentially added to the blocked ELISA plates at 100 μL/well, and allowed to stand for 2 hours at 37° C. The plates were washed with PBST three times, followed by the addition of HRP-labeled mouse anti-human Fc antibodies (1:3000) at 100 μL/well, and the plates were then incubated for 1 hour at 37° C. The plates were washed with PBST three times and then patted on absorbent paper to remove residual droplets as dry as possible, and then, 100 μL of TMB was added to each well, and the plates were left in the dark at room temperature (20±5° C.) for 5 minutes; and 70 μL of 2M H2SO4 stop solution was added to each well to stop the substrate reaction, the OD values were read at 450 nm using a microplate reader, and data analysis and plotting were carried out by GraphPad Prism9 to calculate EC50.

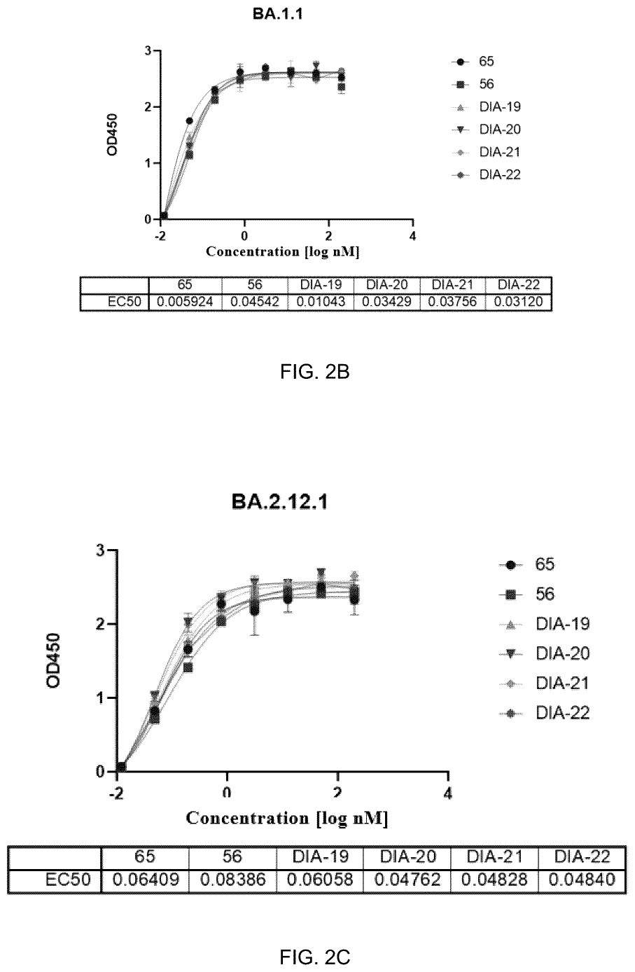

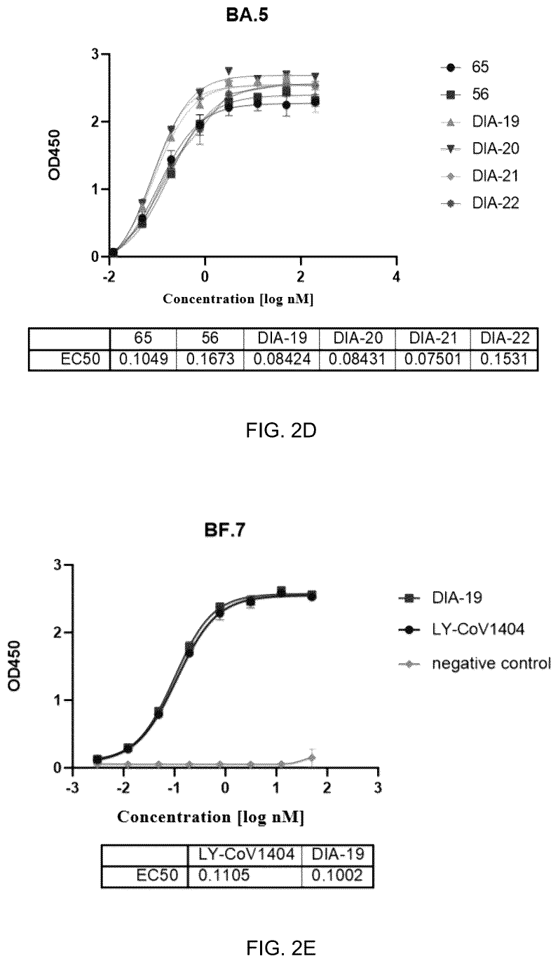

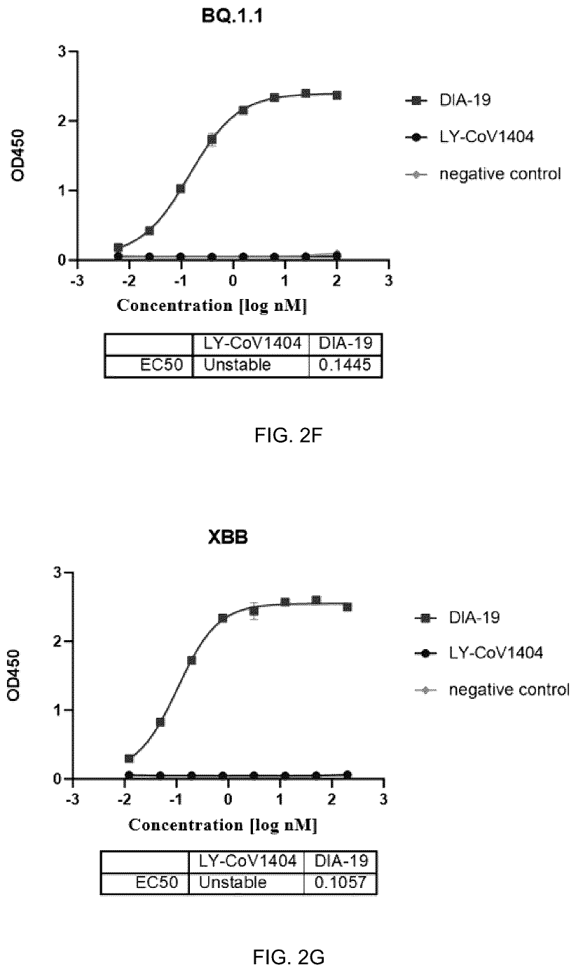

FIGS. 2A to 2G show the binding capacity of the bispecific antibodies and monoclonal antibodies against the novel coronaviruses to RBDs of various novel coronavirus strains. As shown in FIGS. 2A to 2D, the bispecific antibodies DIA-19, DIA-20, DIA-21 and DIA-22 against the novel coronaviruses as well as the parent monoclonal antibodies 65 and 56 all showed concentration-dependent binding to the RBDs of S proteins of the 2019 original strain (2019-nCoV) and the BA.1.1, BA.2.12.1, and BA.5 strains, with comparable affinity.

The results in FIGS. 2E to 2G indicate that DIA-19 binds to the RBDs of S proteins of BF.7, BQ1.1, and XBB strains in a concentration-dependent manner. The control antibody LY-CoV1404 from Eli Lilly shows concentration-dependent binding to the BF.7 strains, but has significantly lost the binding capacity for the recently dominant strains BQ.1.1 and XBB in the United States and Singapore.

### Example 3. Measurement of Affinity Dissociation Constant KD of Bispecific Antibody DIA-19 Against Spike/RBD Proteins of Four Novel Coronaviruses (BA.5, BF.7, BQ.1.1, and XBB)

DIA-19 and LY-CoV1404 were each captured with a chip covalently conjugated with Protein A, both at a concentration of 2 μg/mL and with a contact time of 60 s, a flow rate of 10 μL/min and a regeneration contact time of 30 s. The antigens SARS-CoV-2 (BA.5) Spike RBD, SARS-CoV-2 (BF.7) Spike RBD, SARS-CoV-2 BQ.1.1 (Omicron) Spike RBD, and SARS-CoV-2 XBB (Omicron) Spike RBD were diluted using HBS-NpH7.4 buffer in 2-fold gradients to form 6 concentration gradients, respectively, with the maximum concentrations of 12.5 nM, 6.25 nM, 25 nM, and 6.25 nM respectively, and a zero concentration point was set. The samples were injected on Biacore 8K using 6 M guanidine hydrochloride solution as the regeneration buffer according to the following parameters: binding time: 120 s, dissociation time: 600 s, flow rate: 60 μL/min, and regeneration contact time: 30 s. In the next cycle, the afore-mentioned procedures were repeated. The data was analyzed using Biacore 8K Evaluation Software, as shown in Table 2.

The experimental results indicate that DIA-19 can bind to the RBDs of BA.5, BF.7, BQ.1.1, and XBB with high affinity, with the KD values of 3.59E-11 M, 4.50E-11M, 2.03E-10M, and 8.11E-11M, respectively. The binding affinity of LY-CoV1404 to the RBDs of BA.5 and BF.7 is weaker than that of DIA-19, with the KD values of 1.84E-10M and 1.62E-10M, respectively, and LY-CoV1404 shows no binding signal with BQ.1.1 and XBB.

TABLE 2
Affinity parameters of DIA-19 with
various OMICRON subtype RBDs

| | Virus | ka | kd | KD |
|---|---|---|---|---|
| Antibody | subtype | (1/Ms) | (1/s) | (M) |
| DIA-19 | BA.5 | 1.69E+06 | 6.06E−05 | 3.59E−11 |
| | BF.7 | 1.32E+06 | 5.95E−05 | 4.50E−11 |
| | BQ.1.1 | 1.09E+06 | 2.22E−04 | 2.03E−10 |
| | XBB | 6.59E+06 | 5.35E−04 | 8.11E−11 |
| LY-CoV1404 | BA.5 | 1.44E+06 | 2.64E−04 | 1.84E−10 |
| | BF.7 | 9.10E+05 | 1.48E−04 | 1.62E−10 |
| | BQ.1.1 | / | / | / |
| | XBB | / | / | / |

Note:
/ indicates no binding signal.

### Example 4. Neutralization Experiments of the Bispecific Antibody DIA-19 Against Novel Coronaviruses for Novel Coronavirus Variant Pseudo-Viruses

The bispecific antibody DIA-19 could prevent viral infection by binding to the S protein of the novel coronavirus. Here, the capacity of DIA-19 to block the biological activity of novel coronavirus variants was evaluated using a pseudo-virus model with S proteins expressed on VSV.

Vero cells in good condition were plated in 96-well cell plates and cultured overnight at 37° C. with 5% CO2; the antibodies were diluted to a concentration of 1000 ng/mL with the DMEM medium in a 3-fold concentration gradient, the virus solution diluted in DMEM medium was added to the gradient-diluted antibodies at a volume ratio of 1:1, and the mixture were each incubated for 1 hour in an incubator at 37° C.; then the supernatant was discarded from the Vero cells, followed by the addition of 100 μL of the antibody-virus mixture to the cell plate, which was then cultured for 16 hours at 37° C. with 5% CO2, and then, the count of green fluorescence spots was read with the high-content CQ1; and data analysis and plotting were carried out using Excel and GraphPad Prism 6 to calculate IC50.

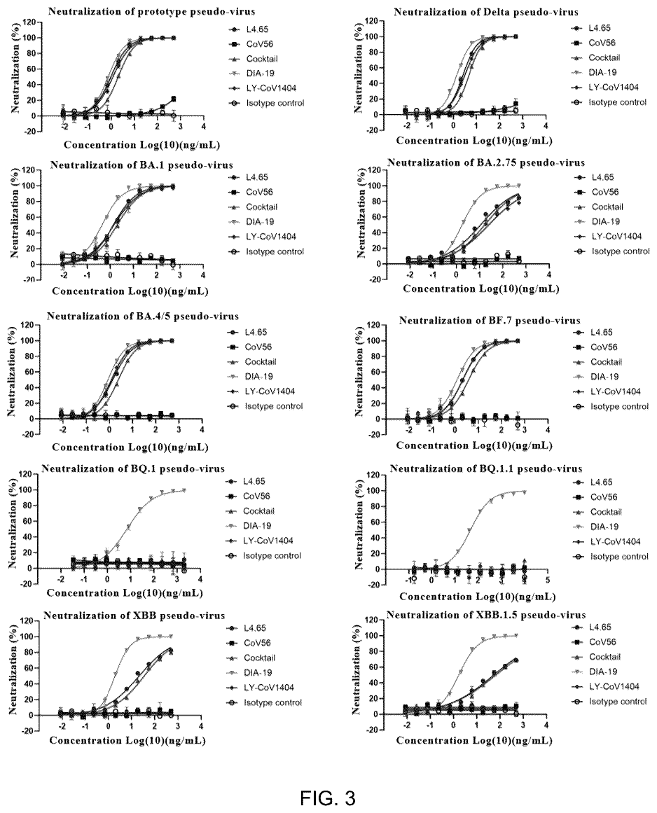

The experimental results are shown in FIG. 3 and Table 3. FIG. 3 shows the infection rate curves and IC50 values of DIA-19, the parental monoclonal antibody, the Cocktail therapy (combination of parental monoclonal antibodies 65 and 56), and the control antibody LY-CoV1404 against different novel coronavirus variants. The experimental results indicate that DIA-19 exhibits good neutralization capacity against the pseudo-viruses of the currently prevalent novel coronavirus strains, and its broad-spectrum and neutralizing capacity against the prevalent novel coronavirus strains is significantly superior to that of the parental monoclonal antibodies 65 (i.e., L4.65) and 56 (i.e., Cov56), and that of the combined administration of the monoclonal antibodies 65 and 56, as well as that of the control antibody LY-CoV1404.

TABLE 3
Neutralization activity (IC50, ng/mL) of DIA-19 against pseudo-viruses of different novel
coronavirus mutant strains

| IC₅₀ (ug/ml) | Prototype | Delta | BA.1 | BA.2.75 | BA.4/5 | BF.7 | BQ.1 | BQ.1.1 | XBB | XBB.1.5 |
|---|---|---|---|---|---|---|---|---|---|---|
| L4.65 | 0.9695 | 2.522 | 1.353 | 11.15 | 1.384 | 2.123 | >2000 | >2000 | 21.4 | 54.12 |
| CoV56 | >2000 | >2000 | >2000 | >2000 | >2000 | >2000 | >2000 | >2000 | >2000 | >2000 |
| Cocktail | 2.006 | 4.607 | 1.992 | 16.91 | 2.423 | 3.819 | >2000 | >2000 | 35.64 | 67.18 |
| DIA-19 | 0.7336 | 1.344 | 0.4233 | 1.759 | 0.8586 | 1.192 | 6.822 | 46.62 | 1.939 | 1.661 |
| LY-CoV1404 | 1.187 | 3.046 | 1.439 | 25.1 | 1.219 | 2.136 | >2000 | >2000 | >2000 | >2000 |

Similar experiments were carried out for DIA-20 to DIA-23, all of which exhibited good neutralization capacity against the pseudo-viruses of the currently prevalent novel coronavirus strains.

### Example 5. Neutralization Experiment of Bispecific Antibody DIA-19 Against Novel Coronavirus for Live Novel Coronavirus Variant Strains

To verify the neutralization activity of DIA-19 against live novel coronavirus variant strains, the live virus neutralization experiments were commissioned to Huawu Biopharmaceutical Technology (Hubei) Co., Ltd. and the National Institute for Viral Disease Control and Prevention (P3 laboratory).

African green monkey kidney passaged cells (VERO-E6) are plated and cultured at 37° C. with 5% CO2 until a monolayer is achieved, and before inoculating the virus in the BSL-3, the cell culture fluid was discarded, and the cells were washed once with the Hanks solution. The antibodies were diluted with the DMEM medium to a concentration of 150 ng/mL in a 3-fold gradient; the viruses were diluted with the DMEM maintenance solution to 200 TCID50/0.1 mL; 0.24 mL of the virus dilution (200 TCID50/0.1 mL) was added to each well of the deep-well plate with the above dilutions, and mixed thoroughly in equal volumes; and the plate was sealed with a sealing film, and then incubated for 1 hour at 37° C. The virus control was prepared by adding 0.24 mL of virus dilution (200 TCID50/0.1 mL) plus 0.24 mL of the maintenance solution, and then, was similarly sealed with the sealing film and incubated for 1 hour at 37° C.; and the normal cell control contained the DMEM maintenance solution only, without viruses added. The Hanks solution in the cell culture wells was discarded. The antibody-virus mixture of each dilution from the 96-well deep plate was seeded onto the monolayer cells, with 4 wells per dilution, 0.1 mL per well, giving the viral load of 100 TCID50 per well. The 96-well culture plate with the sample seeded was cultured in an incubator at 37° C. with 5% CO2. After 3 days of culturing the experimental samples with the virus on the cells, the cell viability was detected using CellTiter Glo chemiluminescence live cell assay kit; 50 μL of CellTiter-Glo reagent was added to each well of the white 96-well plate; then, the plate was gently shaken on a shaker for 2 minutes, and incubated for 10 minutes at room temperature; and the read value of each test well was obtained using a LumiStation 1800 chemiluminescence analyzer. GraphPad Prism9 was used for data analysis, plotting and calculation of IC50.

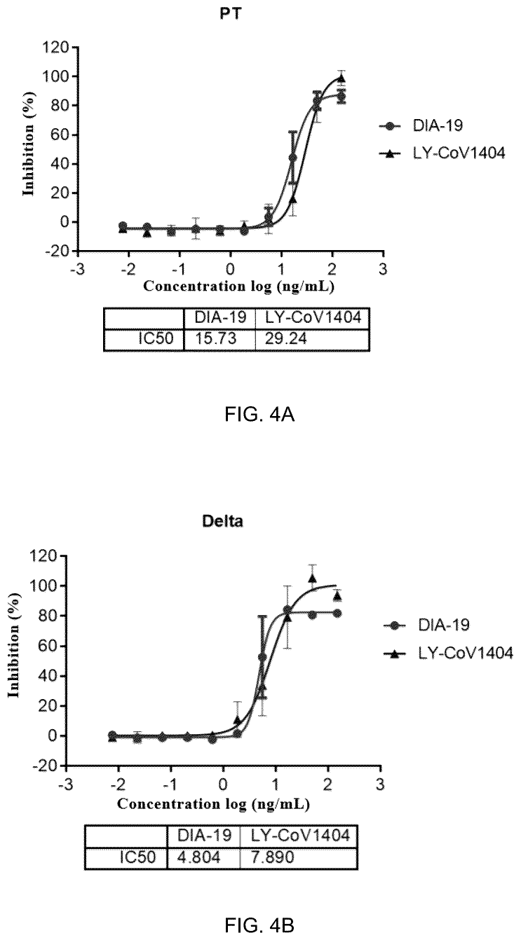

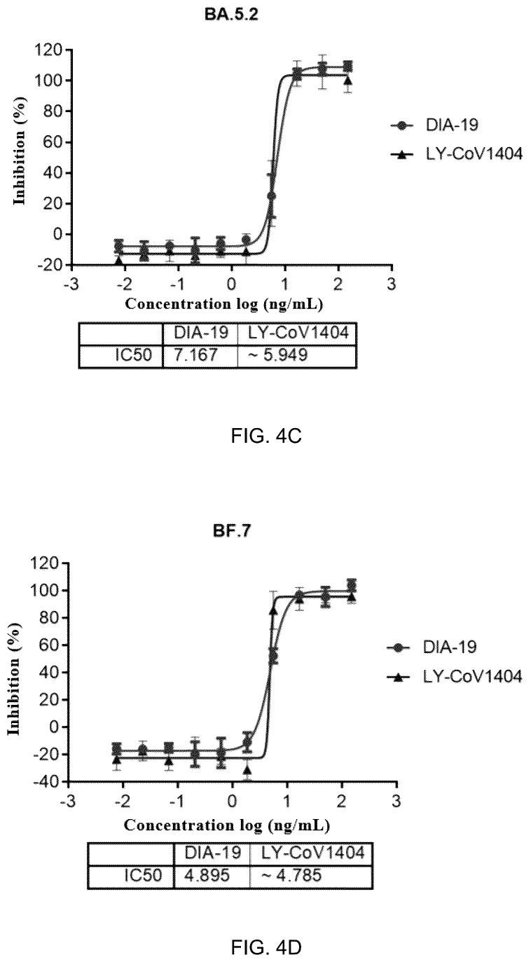

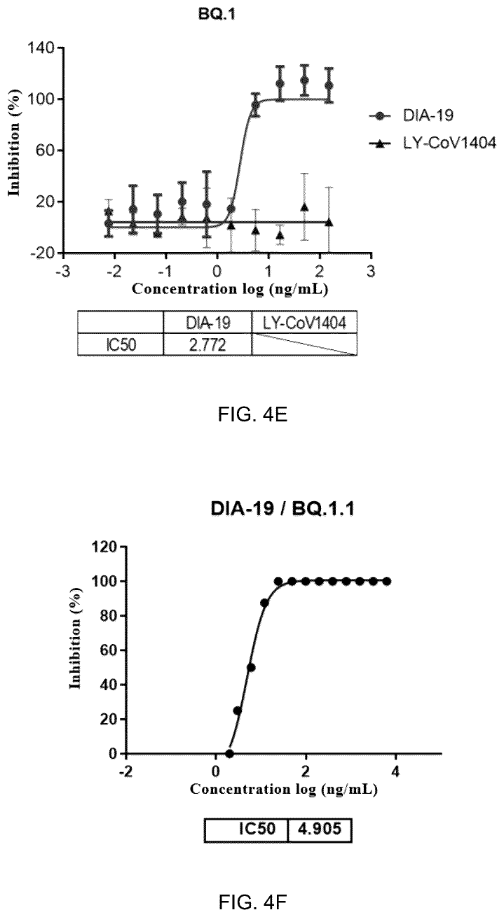

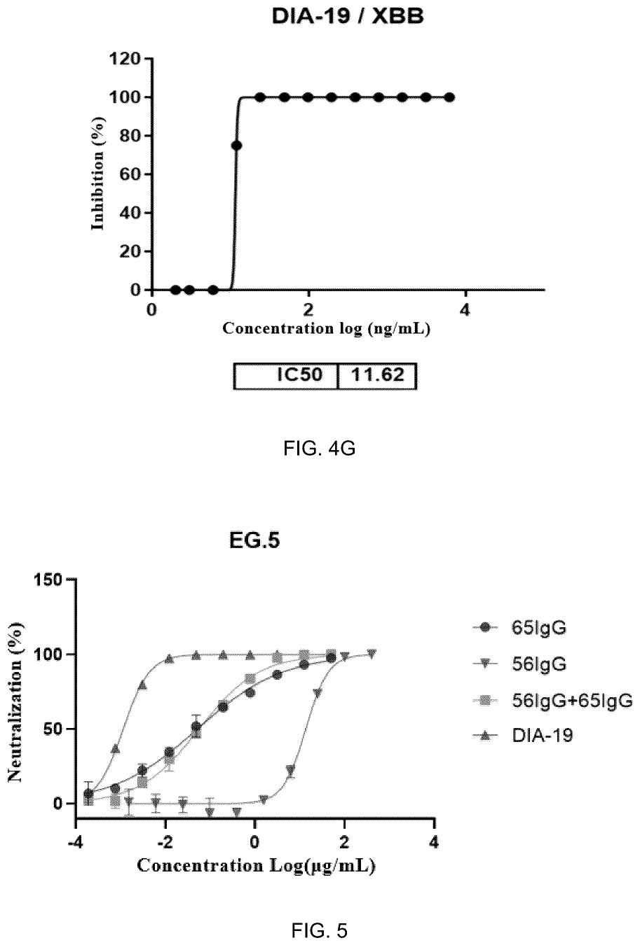

FIGS. 4A to 4G show the cell experiments on the neutralization of various live novel coronavirus strains by DIA-19 and the positive control antibody LY-CoV1404. The experimental results indicate that the DIA-19 molecules can efficiently block the infection of host cells by all the currently tested strains, including 2019-PT (2019-nCov or PT), Delta, BA.5.2 and the latest live viruses B.7, BQ.1, BQ.1.1 and XBB. However, LY-CoV1404 could not block the infection of host cells by live viruses BQ.1, nor could it block the infection of host cells by the live viruses BQ.1.1 and XBB, suggesting the significantly superior broad-spectrum neutralizing capacity of DIA-19 to that of the control antibody LY-CoV1404.

### Example 6. Experiment on Neutralization Effect of Antibody DIA-19 Against SARS-CoV-2 Pseudo-Viruses at Cellular Level

In vitro, Vero cells were infected with the SARS-CoV-2 pseudo-viruses (synthetic recombinant viruses) to measure the inhibitory effects of different concentrations of the antibody on the infection of Vero cells by the pseudo-viruses of novel coronavirus prototype and mutant strains (PT, BA.1, BA.1.1, BA.2, BA.2.75, BA.3, BA.4/5, BF.7, BQ.1, BQ.1.1, XBB, XBB.1.5, and XBB.1.16), to thus test the in vitro broad-spectrum neutralizing activity of the novel coronavirus neutralizing antibody against the pseudo-viruses of different novel coronaviruses. In this experiment, Vero cells were plated in 96-well plates at 1×104 cells/well using a DMEM complete medium containing 2% FBS, and incubated for 24 hours in a carbon dioxide cell incubator at 37° C.; the antibodies diluted in the DMEM medium were fully mixed with the SARS-CoV-2 viruses and incubated for 1 hour at 37° C.; and then the mixture was added to the wells with plated cells. The above 96-well plates were placed in the cell incubator (37° C., 5% CO2) and incubated for 16 hours. After 16 hours, the 96-well plates were taken out and read with the CQ1 high-speed laser confocal high-content flow cytometry analyzer. The inhibition effect on the pseudo-virus infection of cells by the antibodies at different concentrations was calculated based on the GFP fluorescence value.

Based on the results of neutralization inhibition rates at different concentrations, IC50 was calculated by fitting using the biostatistical software GraphPad. The experimental results on the pseudo-virus neutralization of 56IgG, 65IgG, 56IgG+65IgG, and DIA-19 are shown in Table 4.

TABLE 4
Neutralization Effects (IC50 μg/mL) of different forms of antibody against
SARS-COV-2 pseudo-viruses.

| IC50 | PT | BA.1 | BA.1.1 | BA.2 | BA.2.75 | BA.3 | BA.4/5 |
|---|---|---|---|---|---|---|---|
| 56 | 8.375 | 14.79 | 10.82 | 8.316 | 58.81 | 18.5 | 7.529 |
| 65 | 0.0007093 | 0.000394 | 0.0002797 | 0.0003897 | 0.001945 | 0.0004398 | 0.0003769 |
| 65 + 56 | 0.001683 | 0.0006741 | 0.000603 | 0.0006991 | 0.005084 | 0.0007861 | 0.0008157 |
| DIA-19 | 0.0008494 | 0.0002719 | 0.0001819 | 0.0003768 | 0.001008 | 0.0003112 | 0.000332 |

| IC50 | BF.7 | BQ.1 | BQ.1.1 | XBB | XBB.1.5 | XBB.1.16 |
|---|---|---|---|---|---|---|
| 56 | 11.72 | 19.63 | 15.91 | 15.86 | 11.83 | 21.73 |
| 65 | 0.0004464 | 2.093 | 4.584 | 0.003353 | 0.0137 | 0.01033 |
| 65 + 56 | 0.001009 | 21.4 | 37.81 | 0.007901 | 0.02487 | 0.05015 |
| DIA-19 | 0.0004507 | 0.003138 | 0.007511 | 0.000652 | 0.000899 | 0.00072 |

The experimental results indicate that, compared to the 56IgG+65IgG Cocktail method, the bispecific antibody DIA-19 can exhibit a better neutralization effect against SARS-CoV-2 pseudo-viruses. In particular, in the experiments with BQ.1 and BQ.1.1 pseudo-viruses, the neutralization effect of DIA-19 is over 1000 times greater than that of the Cocktail method; and the neutralization effect of DIA-19 against XBB, XBB.1.5, and XBB.1.16 is over 10 times greater than that of the Cocktail method.

### Example 7. Experiment on Neutralization Effect of Antibody DIA-19 Against Pseudo-Viruses of SARS-CoV-2 EG.5 Mutant Strains at Cellular Level

In vitro, Vero cells were infected with the pseudo-viruses (synthetic recombinant viruses) of SARS-CoV-2 EG.5 mutant strains to measure the inhibitory effects of different concentrations of the antibody on the infection of Vero cells by the pseudo-viruses of novel coronavirus EG.5 mutant strains, to thus test the in vitro broad-spectrum neutralizing activity of the novel coronavirus neutralizing antibody against the pseudo-viruses of different novel coronaviruses. In this experiment, Vero cells were plated in 96-well plates at 1×104 cells/well using a DMEM complete medium containing 2% FBS, and incubated for 24 hours in a carbon dioxide cell incubator at 37° C.; the antibodies diluted in the DMEM medium were fully mixed with the SARS-CoV-2 viruses and incubated for 1 hour at 37° C.; and then the mixture was added to the wells with plated cells. The above 96-well plates were placed in the cell incubator (37° C., 5% CO2) and incubated for 16 hours. After 16 hours, the 96-well plates were taken out and read with the CQ1 high-speed laser confocal high-content flow cytometry analyzer. The inhibition effect on the pseudo-virus infection of cells by the antibodies at different concentrations was calculated based on the GFP fluorescence value.

The experimental results on the pseudo-virus neutralization of 56IgG, 65IgG, 56IgG+65IgG, and DIA-19 are shown in Table 5 and FIG. 5.

TABLE 5
Neutralization Effects (IC50 μg/mL) of different forms of antibody
against pseudo-viruses of SARS-CoV-2 EG.5 mutant strains.

| | 56IgG | 65IgG | 56IgG + 65IgG | DIA-19 |
|---|---|---|---|---|
| IC50 | 13.58 | 0.05212 | 0.05634 | 0.001147 |

The experimental results indicate that, compared to the 56IgG+65IgG Cocktail method, the bispecific antibody DIA-19 can exhibit a better neutralization effect against the pseudo-viruses of SARS-CoV-2 EG.5 mutant strains. The neutralization effect of DIA-19 against EG.5 is about 50 times greater than that of the Cocktail method.

### Example 8. Study on the Inhibitory Effect of Antibody DIA-19 Against Novel Coronavirus XBB Strains in Mice

This experiment was aimed at determining the preventive and protective effects of the candidate antibody DIA-19 against SARS-CoV-2 XBB strains in a mouse infection model. In this experiment, K18-hACE2 transgenic mice aged 6-8 weeks were used to evaluate the effect of clearing viruses in the nasal cavities and lungs of the experimental mice after the preventive administration of the candidate antibody DIA-19. A total of 30 mice were set for the preventive experiment and divided into 5 groups (with 6 mice in each group), namely: a 1 mg/kg DIA-19 prevention group (low dose), a 5 mg/kg DIA-19 prevention group (medium dose), a 20 mg/kg DIA-19 prevention group (high dose), a 20 mg/kg LY-cov1404 prevention group, and a 20 mg/kg irrelevant antibody control group. Six hours after intranasal (i.n.) administration of the antibody, the K18-hACE2 mice were infected intra nasally with 2×103 TCID50 of SARS-CoV-2 XBB strains. The viral loads (viral gene copy numbers) in the lungs and nasal cavities of the mice in the prevention groups and their pathological symptoms were detected on Day 3 after the infection.

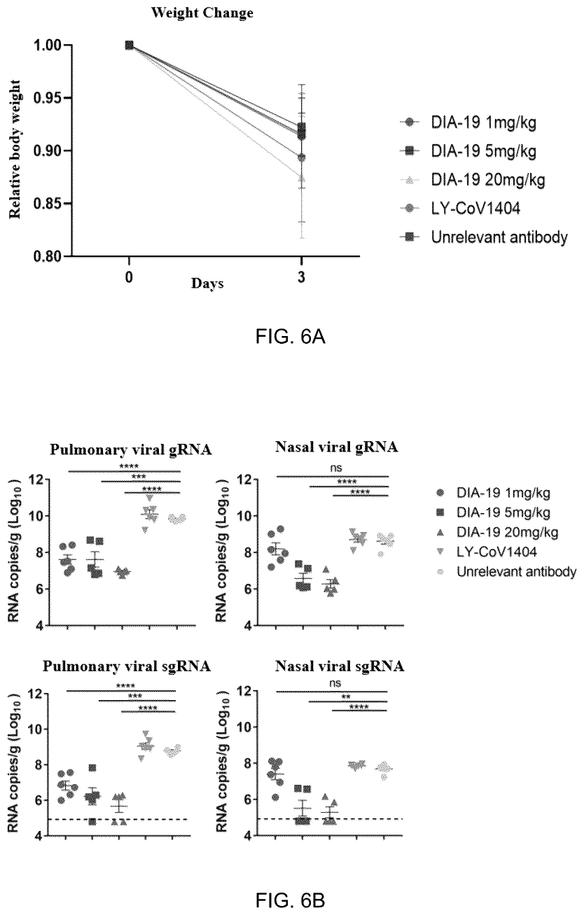

The body weight of the mice was recorded on the day (Day 0) of infection with SARS-CoV-2 XBB strains and on Day 3 after the infection. The detailed results are shown in FIG. 6A.

Based on statistical analysis and the comparison between the 1 mg/kg DIA-19, 5 mg/kg DIA-19 and 20 mg/kg DIA-19 groups with preventive administration via nasal dripping and the irrelevant antibody control group on Day 3 after viral infection, the viral gRNA and sgRNA loads in the lungs were found significantly reduced (as shown in FIG. 6B), with statistical differences observed; the average viral gRNA load in the lungs was reduced by 1.16, 1.16, and 1.82 log 10 copies/g, respectively (as shown in Table 6); and the average viral sgRNA load in the lungs was reduced by 1.95, 2.20, and 2.54 log 10 copies/g, respectively (as shown in Table 8). However, there was no statistical difference in gRNA and sgRNA loads in the lungs between the control antibody LY-cov1404 prevention group and the irrelevant antibody control group.

Based on statistical analysis and the comparison between the 5 mg/kg DIA-19 and 20 mg/kg DIA-19 groups with preventive administration via nasal dripping and the irrelevant antibody control group on Day 3 after viral infection, the viral gRNA and sgRNA loads in the nasal cavities were found significantly reduced (as shown in FIG. 6B), with statistical differences observed; the average viral gRNA load in the nasal cavities was reduced by 2.20 and 2.50 log 10 copies/g, respectively (as shown in Table 7); and the average viral sgRNA load in the nasal cavities was reduced by 2.18 and 2.76 log 10 copies/g, respectively (as shown in Table 9). However, there was no statistical difference in viral gRNA and sgRNA loads in the nasal cavities between the 1 mg/kg DIA-19 prevention group as well as the control antibody LY-cov1404 prevention group and the irrelevant antibody control group.

The above results indicate that the preventive administration of DIA-19 via nasal dripping can effectively inhibit the replication of SARS-CoV-2 XBB strains in the lungs and nasal cavities of the transgenic mice, providing a good protective effect in mice.

TABLE 6
Viral loads (gRNA) in lungs of mice on Day 3 after infection
with SARS-CoV-2 in the prevention experiment

| Group | 1 | 2 | 3 | 4 | 5 |
|---|---|---|---|---|---|
| Disposal | Low-dose | Medium-dose | High-dose | LY-cov1404 | Irrelevant |
| method | DIA-19 | DIA-19 | DIA-19 | | antibody |
| Dose (mg/kg) | 1 | 5 | 20 | 20 | 20 |
| Route of | i.n. | i.n. | i.n. | i.n. | i.n. |
| administration | | | | | |
| Sample | Day 3 after | Day 3 after | Day 3 after | Day 3 after | Day 3 after |
| collection | infection | infection | infection | infection | infection |
| 1 | 8.33 | 8.68 | 6.76 | 10.35 | 9.69 |
| 2 | 7.52 | 7.15 | 7.02 | 9.80 | 9.84 |
| 3 | 6.89 | 6.85 | 7.11 | 9.22 | 9.98 |
| 4 | 7.46 | 6.79 | 6.96 | 9.93 | 9.80 |
| 5 | 8.41 | 8.61 | 6.95 | 10.96 | 9.98 |
| 6 | 7.10 | — | — | 10.31 | 9.76 |
| Mean | 7.62 | 7.62 | 6.96 | 10.10 | 9.84 |
| SD | 0.57 | 0.85 | 0.12 | 0.54 | 0.11 |
| Mean | 1.16 | 1.16 | 1.82 | −1.32 | |
| reduction | | | | | |

Note:
“—” indicates the identified mouse died from non-antibody or non-virus factors, such as choking or overdose anesthesia.

TABLE 7
Viral loads (gRNA) in nasal cavities of mice on Day 3 after
infection with SARS-CoV-2 in the prevention experiment

| Group | 1 | 2 | 3 | 4 | 5 |
|---|---|---|---|---|---|
| Disposal | Low-dose | Medium-dose | High-dose | LY-cov1404 | Irrelevant |
| method | DIA-19 | DIA-19 | DIA-19 | | antibody |
| Dose (mg/kg) | 1 | 5 | 20 | 20 | 20 |
| Route of | i.n. | i.n. | i.n. | i.n. | i.n. |
| administration | | | | | |
| Sample | Day 3 after | Day 3 after | Day 3 after | Day 3 after | Day 3 after |
| collection | infection | infection | infection | infection | infection |
| 1 | 9.01 | 6.12 | 5.77 | 8.10 | 7.92 |
| 2 | 8.15 | 6.18 | 6.03 | 8.93 | 8.64 |
| 3 | 7.58 | 7.12 | 7.09 | 9.13 | 9.95 |
| 4 | 7.95 | 7.38 | 5.99 | 8.52 | 8.46 |
| 5 | 9.29 | 6.08 | 6.50 | 8.83 | 8.94 |
| 6 | 7.20 | — | — | 8.74 | 8.77 |
| Mean | 8.20 | 6.58 | 6.28 | 8.71 | 8.78 |
| SD | 0.74 | 0.56 | 0.47 | 0.33 | 0.61 |
| Mean | 0.58 | 2.20 | 2.50 | 0.07 | |
| reduction | | | | | |

Note:
“—” indicates the identified mouse died from non-antibody or non-virus factors, such as choking or overdose anesthesia.

TABLE 8
Viral loads (sgRNA) in lungs of mice on Day 3 after infection
with SARS-CoV-2 in the prevention experiment

| Group | 1 | 2 | 3 | 4 | 5 |
|---|---|---|---|---|---|
| Disposal | Low-dose | Medium-dose | High-dose | LY-cov1404 | Irrelevant |
| method | DIA-19 | DIA-19 | DIA-19 | | antibody |
| Dose (mg/kg) | 1 | 5 | 20 | 20 | 20 |
| Route of | i.n. | i.n. | i.n. | i.n. | i.n. |
| administration | | | | | |
| Sample | Day 3 after | Day 3 after | Day 3 after | Day 3 after | Day 3 after |
| collection | infection | infection | infection | infection | infection |
| 1 | 7.56 | 7.83 | 6.29 | 9.35 | 8.66 |
| 2 | 6.88 | 6.29 | 6.21 | 8.95 | 8.56 |
| 3 | 5.99 | 6.02 | 6.21 | 8.33 | 8.85 |
| 4 | 6.73 | 6.19 | ND | 8.91 | 8.79 |
| 5 | 7.49 | ND | ND | 9.73 | 9.03 |
| 6 | 6.31 | — | — | 9.06 | 8.80 |
| Mean | 6.83 | 6.58 | 6.24 | 9.06 | 8.78 |
| SD | 0.57 | 0.73 | 0.04 | 0.43 | 0.15 |
| Mean | 1.95 | 2.20 | 2.54 | −0.28 | |
| reduction | | | | | |

Note:
“—” indicates the identified mouse died from non-antibody or non-virus factors, such as choking or overdose anesthesia; and “ND” indicates not detected.

TABLE 9
Viral loads (sgRNA) in nasal cavities of mice on Day 3 after
infection with SARS-CoV-2 in the prevention experiment

| Group | 1 | 2 | 3 | 4 | 5 |
|---|---|---|---|---|---|
| Disposal | Low-dose | Medium-dose | High-dose | LY-cov1404 | Irrelevant |
| method | DIA-19 | DIA-19 | DIA-19 | | antibody |
| Dose (mg/kg) | 1 | 5 | 20 | 20 | 20 |
| Route of | i.n. | i.n. | i.n. | i.n. | i.n. |
| administration | | | | | |
| Sample | Day 3 after | Day 3 after | Day 3 after | Day 3 after | Day 3 after |
| collection | infection | infection | infection | infection | infection |
| 1 | 8.08 | ND | ND | 7.87 | 7.71 |
| 2 | 7.37 | ND | 6.18 | 7.96 | 7.96 |
| 3 | 7.79 | 6.60 | ND | 7.70 | 7.23 |
| 4 | 6.94 | 6.60 | ND | 7.87 | 7.81 |
| 5 | 8.12 | ND | 5.86 | 7.85 | 7.71 |
| 6 | 6.11 | — | — | 7.90 | 7.67 |
| Mean | 7.40 | 6.60 | 6.02 | 7.86 | 7.68 |
| SD | 0.71 | 0.00 | 0.16 | 0.08 | 0.22 |
| Mean | 1.38 | 2.18 | 2.76 | 0.92 | |
| reduction | | | | | |

Note:
“—” indicates the identified mouse died from non-antibody or non-virus factors, such as choking or overdose anesthesia; and “ND” indicates not detected.

### Example 9. Evaluation on the Therapeutic Effect of Injection Administration of Antibody DIA-19 Against Novel Coronavirus XBB Strains in Mice

This experiment was aimed at evaluating the therapeutic effect of the candidate antibody DIA-19 against SARS-CoV-2 XBB strains in a mouse infection model. In this experiment, K18-hACE2 transgenic mice aged 6-8 weeks were infected with SARS-CoV-2 XBB strains and then intraperitoneally (i.p.) injected with the candidate antibody DIA-19 to test the effect of clearing viruses in the nasal cavities and lungs of the experimental mice. A total of 24 mice were set for the intraperitoneal injection treatment experiment and divided into 4 groups (with 6 mice in each group), namely: a 10 mg/kg DIA-19 treatment group, a 25 mg/kg DIA-19 treatment group, a 50 mg/kg DIA-19 treatment group and a PBS control group. The transgenic mice were infected with 1.5×104 TCID50 of SARS-CoV-2 XBB strains via nasal dripping; after two hours, different concentrations of DIA-19 antibodies were injected intraperitoneally for treatment, respectively; and 24 hours after the first treatment, the DIA-19 antibodies were re-injected for treatment. On Day 4 after viral infection, the mice were euthanized, and the lung and turbinate tissues were harvested to detect the viral loads (viral genome copy numbers) in the lungs and nasal cavities of the mice and their pathological symptom.

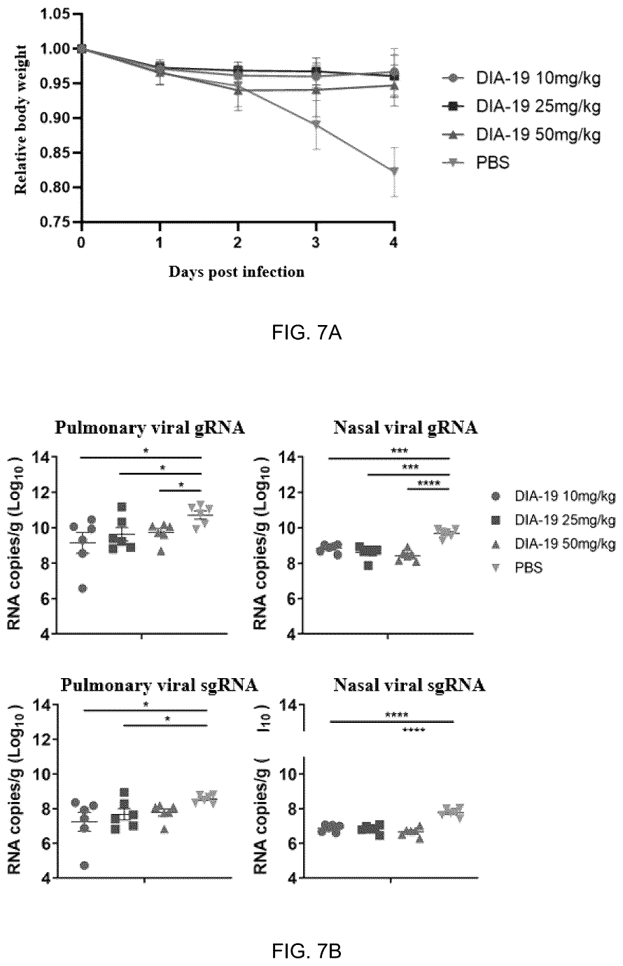

The body weight of the mice was recorded on the day (Day 0) of infection with SARS-CoV-2 XBB strains and for the subsequent 4 days after the infection. The detailed results are shown in FIG. 7A.

Based on statistical analysis and the comparison between the 10 mg/kg, 25 mg/kg and 50 mg/kg DIA-19 antibody groups with therapeutic intraperitoneal administration and the PBS control group on Day 4 after viral infection, the viral gRNA and sgRNA loads in the lungs were found significantly reduced (as shown in FIG. 7B), with statistical differences observed; the average viral gRNA load in the lungs was reduced by 1.57, 1.08 and 0.98 log 10 copies/g, respectively (as shown in Table 10); and the average viral sgRNA load in the lungs was reduced by 1.31, 0.88 and 0.77 log 10 copies/g copies/g, respectively (as shown in Table 12).

Based on statistical analysis and the comparison between the 10 mg/kg, 25 mg/kg and 50 mg/kg DIA-19 antibody groups with therapeutic intraperitoneal administration and the PBS control group on Day 4 after viral infection, the viral gRNA and sgRNA loads in the nasal cavities were found significantly reduced (as shown in FIG. 7B), with statistical differences observed; the average viral gRNA load in the nasal cavities was reduced by 0.85, 1.10 and 1.25 log 10 copies/g, respectively (as shown in Table 11); and the average viral sgRNA load in the nasal cavities was reduced by 0.90, 0.95 and 1.11 log 10 copies/g, respectively (as shown in Table 13).

The above results indicate that the administration of DIA-19 antibodies via intraperitoneal injection can effectively inhibit the replication of SARS-CoV-2 XBB strains in the lungs and nasal cavities of the transgenic mice, providing a good protective effect in mice.

TABLE 10
Viral loads (gRNA) in lungs of mice on Day 4 after infection with
SARS-CoV-2 in the intraperitoneal injection treatment experiment

| Group | 1 | 2 | 3 | 4 |
|---|---|---|---|---|
| Disposal | Low-dose | Medium-dose | High-dose | PBS |
| method | DIA-19 | DIA-19 | DIA-19 | |
| Dose (mg/kg) | 10 | 25 | 50 | |
| Route of | i.p. | i.p. | i.p. | i.p. |
| administration | | | | |
| Sample | Day 4 | Day 4 | Day 4 | Day 4 |
| collection | after | after | after | after |
| | infection | infection | infection | infection |
| 1 | 8.55 | 8.89 | 10.18 | 11.04 |
| 2 | 9.93 | 8.80 | 8.70 | 9.90 |
| 3 | 9.30 | 10.33 | 10.10 | 11.11 |
| 4 | 10.06 | 11.17 | 9.72 | 10.77 |
| 5 | 6.57 | 9.22 | 10.07 | 10.21 |
| 6 | 10.46 | 9.38 | 9.62 | 11.28 |
| Mean | 9.14 | 9.63 | 9.73 | 10.71 |
| SD | 1.30 | 0.84 | 0.50 | 0.50 |
| Mean reduction | 1.57 | 1.08 | 0.98 | |

TABLE 11
Viral loads (gRNA) in nasal cavities of mice on
Day 4 after infection with SARS-CoV-2 in the
intraperitoneal injection treatment experiment

| Group | 1 | 2 | 3 | 4 |
|---|---|---|---|---|
| Disposal | Low-dose | Medium-dose | High-dose | PBS |
| method | DIA-19 | DIA-19 | DIA-19 | |
| Dose (mg/kg) | 10 | 25 | 50 | |
| Route of | i.p. | i.p. | i.p. | i.p. |
| administration | | | | |
| Sample | Day 4 | Day 4 | Day 4 | Day 4 |
| collection | after | after | after | after |
| | infection | infection | infection | infection |
| 1 | 8.93 | 8.74 | 8.16 | 9.93 |
| 2 | 8.47 | 7.87 | 8.40 | 9.25 |
| 3 | 8.87 | 8.92 | 8.56 | 9.54 |
| 4 | 8.68 | 8.74 | 8.48 | 9.76 |
| 5 | 9.03 | 8.66 | 8.10 | 9.73 |
| 6 | 9.04 | 8.64 | 8.92 | 9.92 |
| Mean | 8.84 | 8.59 | 8.44 | 9.69 |
| SD | 0.20 | 0.33 | 0.27 | 0.24 |
| Mean reduction | 0.85 | 1.10 | 1.25 | |

TABLE 12
Viral loads (sgRNA) in lungs of mice on Day 4 after infection with
SARS-CoV-2 in the intraperitoneal injection treatment experiment

| Group | 1 | 2 | 3 | 4 |
|---|---|---|---|---|
| Disposal | Low-dose | Medium-dose | High-dose | PBS |
| method | DIA-19 | DIA-19 | DIA-19 | |
| Dose (mg/kg) | 10 | 25 | 50 | |
| Route of | i.p. | i.p. | i.p. | i.p. |
| administration | | | | |
| Sample | Day 4 | Day 4 | Day 4 | Day 4 |
| collection | after | after | after | after |
| | infection | infection | infection | infection |
| 1 | 6.87 | 7.01 | 8.09 | 8.67 |
| 2 | 7.94 | 6.81 | 6.84 | 8.32 |
| 3 | 7.44 | 8.28 | 8.19 | 8.79 |
| 4 | 8.18 | 8.94 | 7.76 | 8.49 |
| 5 | 4.73 | 7.40 | 8.10 | 8.27 |
| 6 | 8.35 | 7.65 | 7.79 | 8.80 |
| Mean | 7.25 | 7.68 | 7.79 | 8.56 |
| SD | 1.23 | 0.73 | 0.46 | 0.21 |
| Mean reduction | 1.31 | 0.88 | 0.77 | |

TABLE 13
Viral loads (sgRNA) in nasal cavities of mice on
Day 4 after infection with SARS-CoV-2 in the intraperitoneal
injection treatment experiment

| Group | 1 | 2 | 3 | 4 |
|---|---|---|---|---|
| Disposal | Low-dose | Medium-dose | High-dose | PBS |
| method | DIA-19 | DIA-19 | DIA-19 | |
| Dose (mg/kg) | 10 | 25 | 50 | |
| Route of | i.p. | i.p. | i.p. | i.p. |
| administration | | | | |
| Sample | Day 4 | Day 4 | Day 4 | Day 4 |
| collection | after | after | after | after |
| | infection | infection | infection | infection |
| 1 | 6.89 | 6.79 | 6.51 | 8.03 |
| 2 | 6.67 | 6.45 | 6.72 | 7.42 |
| 3 | 7.05 | 6.99 | 6.73 | 7.64 |
| 4 | 6.60 | 6.86 | 6.75 | 7.83 |
| 5 | 7.08 | 7.09 | 6.28 | 7.75 |
| 6 | 7.00 | 6.81 | 7.02 | 8.00 |
| Mean | 6.88 | 6.83 | 6.67 | 7.78 |
| SD | 0.19 | 0.20 | 0.23 | 0.21 |
| Mean reduction | 0.90 | 0.95 | 1.11 | |

### Example 10. Study on the Inhibitory Effect of Antibody DIA-19 Against Novel Coronavirus Delta Strains in Mice

This experiment was aimed at determining the preventive, therapeutic and protective effects of the candidate antibody DIA-19 against SARS-CoV-2 Delta strains in a mouse infection model. In this experiment, K18-hACE2 transgenic mice aged 6-8 weeks were used to evaluate the effect of clearing viruses in the brains and lungs of the experimental mice after the preventive and therapeutic administration of the candidate antibody DIA-19.

A total of 30 mice were set for the preventive experiment and divided into 5 groups (with 6 mice in each group), namely: a 0.4 mg/kg DIA-19 prevention group, a 2 mg/kg DIA-19 prevention group, a 10 mg/kg DIA-19 prevention group, a 10 mg/kg LY-cov1404 prevention group, and a 10 mg/kg irrelevant antibody control group. Six hours after administration of the antibody via nasal dripping, the transgenic mice were infected intra nasally with 1×104 TCID50 of SARS-CoV-2 Delta strains.

A total of 30 mice were set for the treatment experiment and divided into 5 groups (with 6 mice in each group), namely: a 5 mg/kg DIA-19 treatment group, a 10 mg/kg DIA-19 treatment group, a 30 mg/kg DIA-19 treatment group, a 30 mg/kg LY-cov1404 treatment group, and a 30 mg/kg irrelevant antibody control group. The transgenic mice were intra nasally infected with 1×104 TCID50 of SARS-CoV-2 Delta strains, and then subjected to intraperitoneal injection after 2 hours. The viral loads (viral gene copy numbers) in the brains and lungs of the mice in the prevention and treatment groups and their pathological symptoms were detected on Day 3 after the infection.

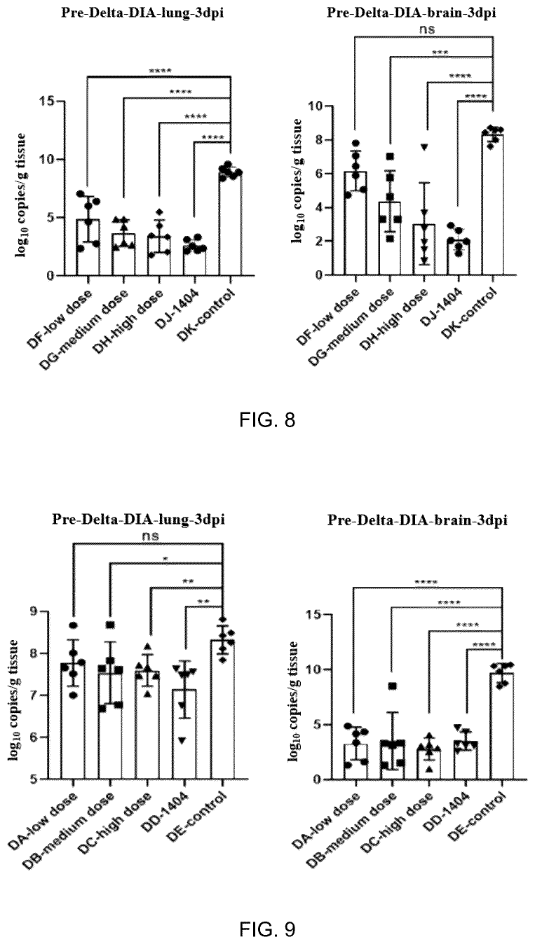

The results of the preventive experiment show (FIG. 8) that, based on the comparison between the 0.4 mg/kg DIA-19, 2 mg/kg DIA-19, 10 mg/kg DIA-19, and 10 mg/kg control antibody LY-cov1404 groups with preventive administration via nasal dripping and the irrelevant antibody control group on Day 3 after viral infection, the average viral load in the lungs was reduced by 4.05, 5.25, 5.51, and 6.31 log 10 copies/g, respectively, with statistical differences observed. Based on the comparison between the 2 mg/kg DIA-19, 10 mg/kg DIA-19, and 10 mg/kg control antibody LY-cov1404 groups with preventive administration via nasal dripping and the irrelevant antibody control group on Day 3 after viral infection, the average viral load in the brains was reduced by 3.96, 5.29 and 6.23 log 10 copies/g, respectively, with statistical differences observed.

The results of the therapeutic experiment show (FIG. 9) that, based on the comparison between the 10 mg/kg DIA-19, 30 mg/kg DIA-19, and 30 mg/kg control antibody LY-cov1404 treatment groups and the irrelevant antibody control group on Day 3 after viral infection, the average viral load in the lungs was reduced by 0.78, 0.73, and 1.18 log 10 copies/g, respectively, with statistical differences observed. Based on the comparison between the 5 mg/kg DIA-19, 10 mg/kg DIA-19, 30 mg/kg DIA-19, and 30 mg/kg control antibody LY-LYcov1404 treatment groups and the irrelevant antibody control group, the average viral load in the brains was reduced by 6.41, 6.18, 6.91, and 6.19 log 10 copies/g, respectively, with statistical differences observed.

The above results indicate that DIA-19 has good preventive, therapeutic and protective effects against SARS-CoV-2 Delta strains in the mouse infection model.

The antibody sequence of the present disclosure is as follows:

| SEQ ID NO. 1 DIA-19 | |
|---|---|
| QITLKESGPTLVKPTQTLTLTCTFSGFSLSTSGVGVAWIRQPPGKALEWLALIYWDNDKRSSP | |
| SLNNRLTITKDTSKNQVVLTMTNMDPEDTATYYCAHFFSHYDSSNYYYGSWFDPWGQGTLV | |
| 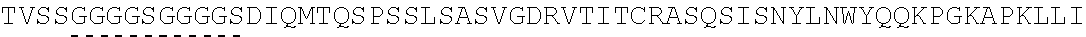 | |
| 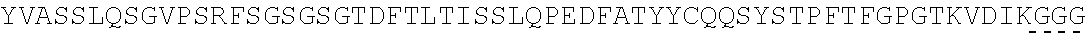 | |
| 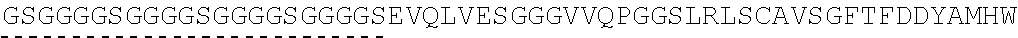 | |
| VRQAPGKGLDWVSLISGDGSYTYYADSVKGRFTISRDSSKNSLYLQMNSLRTEDTALYYCAK | |
| 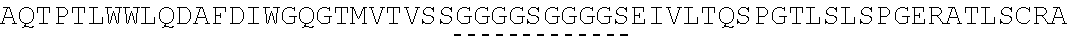 | |
| SQSFDSRYLGWYQQKSGQAPRLLIYGASSRATGIPDRFSGSGSGTDFTLTISRLEPEDFAVYY | |
| 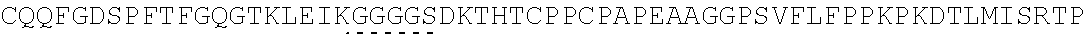 | |
| EVTCVVVDVSHEDPEVKFNWYVDGVEVHNAKTKPREEQYNSTYRVVSVLTVLHQDWLNGK | |
| EYKCKVSNKALPAPIEKTISKAKGQPREPQVYTLPPSREEMTKNQVSLTCLVKGFYPSDIAVE | |
| WESNGQPENNYKTTPPVLDSDGSFFLYSKLTVDKSRWQQGNVFSCSVMHEALHNHYTQKS | |
| LSLSPGK | |
| SEQ ID NO. 2 DIA-20 | |
| EIVLTQSPGTLSLSPGERATLSCRASQSFDSRYLGWYQQKSGQAPRLLIYGASSRATGIPDRF | |
| SGSGSGTDFTLTISRLEPEDFAVYYCQQFGDSPFTFGQGTKLEIKGGGGSGGGGSEVQLVES | |
| GGGVVQPGGSLRLSCAVSGFTFDDYAMHWVRQAPGKGLDWVSLISGDGSYTYYADSVKGR | |
| FTISRDSSKNSLYLQMNSLRTEDTALYYCAKAQTPTLWWLQDAFDIWGQGTMVTVSSGGGG | |
| SGGGGSGGGGSGGGGSGGGGSDIQMTQSPSSLSASVGDRVTITCRASQSISNYLNWYQQ | |
| KPGKAPKLLIYVASSLQSGVPSRFSGSGSGTDFTLTISSLQPEDFATYYCQQSYSTPFTFGPG | |
| TKVDIKGGGGSGGGGSQITLKESGPTLVKPTQTLTLTCTFSGFSLSTSGVGVAWIRQPPGKAL | |
| EWLALIYWDNDKRSSPSLNNRLTITKDTSKNQVVLTMTNMDPEDTATYYCAHFFSHYDSSNY | |
| YYGSWFDPWGQGTLVTVSSGGGGSDKTHTCPPCPAPEAAGGPSVFLFPPKPKDTLMISRTP | |
| EVTCVVVDVSHEDPEVKFNWYVDGVEVHNAKTKPREEQYNSTYRVVSVLTVLHQDWLNGK | |
| EYKCKVSNKALPAPIEKTISKAKGQPREPQVYTLPPSREEMTKNQVSLTCLVKGFYPSDIAVE | |
| WESNGQPENNYKTTPPVLDSDGSFFLYSKLTVDKSRWQQGNVFSCSVMHEALHNHYTQKS | |
| LSLSPGK | |
| SEQ ID NO. 3 DIA-21 | |
| EVQLVESGGGVVQPGGSLRLSCAVSGFTFDDYAMHWVRQAPGKGLDWVSLISGDGSYTYY | |
| ADSVKGRFTISRDSSKNSLYLQMNSLRTEDTALYYCAKAQTPTLWWLQDAFDIWGQGTMVT | |
| VSSGGGGSGGGGSEIVLTQSPGTLSLSPGERATLSCRASQSFDSRYLGWYQQKSGQAPRLL | |
| IYGASSRATGIPDRFSGSGSGTDFTLTISRLEPEDFAVYYCQQFGDSPFTFGQGTKLEIKGGG | |
| GSGGGGSGGGGSGGGGSGGGGSQITLKESGPTLVKPTQTLTLTCTFSGFSLSTSGVGVAWI | |
| RQPPGKALEWLALIYWDNDKRSSPSLNNRLTITKDTSKNQVVLTMTNMDPEDTATYYCAHFF | |
| SHYDSSNYYYGSWFDPWGQGTLVTVSSGGGGSGGGGSDIQMTQSPSSLSASVGDRVTITC | |
| RASQSISNYLNWYQQKPGKAPKLLIYVASSLQSGVPSRFSGSGSGTDFTLTISSLQPEDFATY | |
| YCQQSYSTPFTFGPGTKVDIKGGGGSDKTHTCPPCPAPEAAGGPSVFLFPPKPKDTLMISRT | |
| PEVTCVVVDVSHEDPEVKFNWYVDGVEVHNAKTKPREEQYNSTYRVVSVLTVLHQDWLNG | |
| KEYKCKVSNKALPAPIEKTISKAKGQPREPQVYTLPPSREEMTKNQVSLTCLVKGFYPSDIAVE | |
| WESNGQPENNYKTTPPVLDSDGSFFLYSKLTVDKSRWQQGNVFSCSVMHEALHNHYTQKS | |
| LSLSPGK | |
| SEQ ID NO. 4 DIA-22 | |
| DIQMTQSPSSLSASVGDRVTITCRASQSISNYLNWYQQKPGKAPKLLIYVASSLQSGVPSRFS | |
| GSGSGTDFTLTISSLQPEDFATYYCQQSYSTPFTFGPGTKVDIKGGGGSGGGGSQITLKESG | |
| PTLVKPTQTLTLTCTFSGFSLSTSGVGVAWIRQPPGKALEWLALIYWDNDKRSSPSLNNRLTIT | |
| KDTSKNQVVLTMTNMDPEDTATYYCAHFFSHYDSSNYYYGSWFDPWGQGTLVTVSSGGGG | |
| SGGGGSGGGGSGGGGSGGGGSEIVLTQSPGTLSLSPGERATLSCRASQSFDSRYLGWYQ | |
| QKSGQAPRLLIYGASSRATGIPDRFSGSGSGTDFTLTISRLEPEDFAVYYCQQFGDSPFTFGQ | |
| GTKLEIKGGGGSGGGGSEVQLVESGGGVVQPGGSLRLSCAVSGFTFDDYAMHWVRQAPGK | |
| GLDWVSLISGDGSYTYYADSVKGRFTISRDSSKNSLYLQMNSLRTEDTALYYCAKAQTPTLW | |
| WLQDAFDIWGQGTMVTVSSGGGGSDKTHTCPPCPAPEAAGGPSVFLFPPKPKDTLMISRTP | |
| EVTCVVVDVSHEDPEVKFNWYVDGVEVHNAKTKPREEQYNSTYRVVSVLTVLHQDWLNGK | |
| EYKCKVSNKALPAPIEKTISKAKGQPREPQVYTLPPSREEMTKNQVSLTCLVKGFYPSDIAVE | |
| WESNGQPENNYKTTPPVLDSDGSFFLYSKLTVDKSRWQQGNVFSCSVMHEALHNHYTQKS | |
| LSLSPGK | |
| SEQ ID NO. 5 CoV56 light chain | |
| DIQMTQSPSSLSASVGDRVTITCRASQSISNYLNWYQQKPGKAPKLLIYVASSLQSGVPSRFS | |
| GSGSGTDFTLTISSLQPEDFATYYCQQSYSTPFTFGPGTKVDIKRTVAAPSVFIFPPSDEQLKS | |
| GTASVVCLLNNFYPREAKVQWKVDNALQSGNSQESVTEQDSKDSTYSLSSTLTLSKADYEK | |
| HKVYACEVTHQGLSSPVTKSFNRGECS | |
| SEQ ID NO. 6 CoV56 heavy chain | |
| EVQLVESGGGVVQPGGSLRLSCAVSGFTFDDYAMHWVRQAPGKGLDWVSLISGDGSYTYY | |
| ADSVKGRFTISRDSSKNSLYLQMNSLRTEDTALYYCAKAQTPTLWWLQDAFDIWGQGTMVT | |
| VSSASTKGPSVFPLAPSSKSTSGGTAALGCLVKDYFPEPVTVSWNSGALTSGVHTFPAVLQS | |
| SGLYSLSSVVTVPSSSLGTQTYICNVNHKPSNTKVDKKVEPKSCDKTHTCPPCPAPELLGGP | |
| SVFLFPPKPKDTLMISRTPEVTCVVVDVSHEDPEVKFNWYVDGVEVHNAKTKPREEQYNSTY | |
| RVVSVLTVLHQDWLNGKEYKCKVSNKALPAPIEKTISKAKGQPREPQVYTLPPSREEMTKNQ | |
| VSLTCLVKGFYPSDIAVEWESNGQPENNYKTTPPVLDSDGSFFLYSKLTVDKSRWQQGNVFS | |
| CSVMHEALHNHYTQKSLSLSPGK | |
| SEQ ID NO. 7 L4.65 light chain | |
| EIVLTQSPGTLSLSPGERATLSCRASQSFDSRYLGWYQQKSGQAPRLLIYGASSRATGIPDRF | |
| SGSGSGTDFTLTISRLEPEDFAVYYCQQFGDSPFTFGQGTKLEIKRTVAAPSVFIFPPSDEQLK | |
| SGTASVVCLLNNFYPREAKVQWKVDNALQSGNSQESVTEQDSKDSTYSLSSTLTLSKADYE | |
| KHKVYACEVTHQGLSSPVTKSFNRGECS | |
| SEQ ID NO. 8 L4.65 heavy chain | |
| QITLKESGPTLVKPTQTLTLTCTFSGFSLSTSGVGVAWIRQPPGKALEWLALIYWDNDKRSSP | |
| SLNNRLTITKDTSKNQVVLTMTNMDPEDTATYYCAHFFSHYDSSNYYYGSWFDPWGQGTLV | |
| TVSSASTKGPSVFPLAPSSKSTSGGTAALGCLVKDYFPEPVTVSWNSGALTSGVHTFPAVLQ | |
| SSGLYSLSSVVTVPSSSLGTQTYICNVNHKPSNTKVDKRVEPKSCDKTHTCPPCPAPELLGG | |
| PSVFLFPPKPKDTLMISRTPEVTCVVVDVSHEDPEVKFNWYVDGVEVHNAKTKPREEQYNST | |
| YRVVSVLTVLHQDWLNGKEYKCKVSNKALPAPIEKTISKAKGQPREPQVYTLPPSRDELTKNQ | |
| VSLTCLVKGFYPSDIAVEWESNGQPENNYKTTPPVLDSDGSFFLYSKLTVDKSRWQQGNVFS | |
| CSVMHEALHNHYTQKSLSLSPGK | |
| SEQ ID NO. 9 LY-CoV1404 light chain | |
| QSALTQPASVSGSPGQSITISCTATSSDVGDYNYVSWYQQHPGKAPKLMIFEVSDRPSGISN | |
| RFSGSKSGNTASLTISGLQAEDEADYYCSSYTTSSAVFGGGTKLTVLGQPKAAPSVTLFPPSS | |
| EELQANKATLVCLISDFYPGAVTVAWKADSSPVKAGVETTTPSKQSNNKYAASSYLSLTPEQ | |
| WKSHRSYSCQVTHEGSTVEKTVAPTECS | |
| SEQ ID NO. 10 LY-CoV1404 heavy chain | |
| QITLKESGPTLVKPTQTLTLTCTFSGFSLSISGVGVGWLRQPPGKALEWLALIYWDDDKRYSP | |
| SLKSRLTISKDTSKNQVVLKMTNIDPVDTATYYCAHHSISTIFDHWGQGTLVTVSSASTKGPSV | |
| FPLAPSSKSTSGGTAALGCLVKDYFPEPVTVSWNSGALTSGVHTFPAVLQSSGLYSLSSVVTV | |
| PSSSLGTQTYICNVNHKPSNTKVDKRVEPKSCDKTHTCPPCPAPELLGGPSVFLFPPKPKDT | |
| LMISRTPEVTCVVVDVSHEDPEVKFNWYVDGVEVHNAKTKPREEQYNSTYRVVSVLTVLHQ | |
| DWLNGKEYKCKVSNKALPAPIEKTISKAKGQPREPQVYTLPPSREEMTKNQVSLTCLVKGFY | |
| PSDIAVEWESNGQPENNYKTTPPVLDSDGSFFLYSKLTVDKSRWQQGNVFSCSVMHEALHN | |
| HYTQKSLSLSPGK | |
| SEQ ID NO. 11 | monoclonal antibody 65 HCDR1 TSGVGVA |
| SEQ ID NO. 12 | monoclonal antibody 65 HCDR2 LIYWDNDKRSSPSLNN |
| SEQ ID NO. 13 | monoclonal antibody 65 HCDR3 FFSHYDSSNYYYGSWFDP |
| SEQ ID NO. 14 | monoclonal antibody 65 LCDR1 RASQSFDSRYLG |
| SEQ ID NO. 15 | monoclonal antibody 65 LCDR2GASSRAT |
| SEQ ID NO. 16 | monoclonal antibody 65 LCDR3QQFGDSPFT |
| SEQ ID NO. 17 | monoclonal antibody 56 HCDR1 DYAMH |
| SEQ ID NO. 18 | monoclonal antibody 56 HCDR2 LISGDGSYTYYADSVKG |
| SEQ ID NO. 19 | monoclonal antibody 56 HCDR3 AQTPTLWWLQDAFDI |
| SEQ ID NO. 20 | monoclonal antibody 56 LCDR1 RASQSISNYLN |
| SEQ ID NO. 21 | monoclonal antibody 56 LCDR2VASSLQS |
| SEQ ID NO. 22 | monoclonal antibody 56 LCDR3QQSYSTPFT |
| SEQ ID NO. 23 | monoclonal antibody 65 VH |

QITLKESGPTLVKPTQTLTLTCTFSGFSLSTSGVGVAWIRQPPGKALEWLALIYWDNDKRSSP
SLNNRLTITKDTSKNQVVLTMTNMDPEDTATYYCAHFFSHYDSSNYYYGSWFDPWGQGTLV
SEQ ID NO. 24 monoclonal antibody 65 VL
EIVLTQSPGTLSLSPGERATLSCRASQSFDSRYLGWYQQKSGQAPRLLIYGASSRATGIPDRF
SGSGSGTDFTLTISRLEPEDFAVYYCQQFGDSPFTFGQGTKLEIK
SEQ ID NO. 25 monoclonal antibody 56 VH
EVQLVESGGGVVQPGGSLRLSCAVSGFTFDDYAMHWVRQAPGKGLDWVSLISGDGSYTYY
ADSVKGRFTISRDSSKNSLYLQMNSLRTEDTALYYCAKAQTPTLWWLQDAFDIWGQGTMVT
VSS
SEQ ID NO. 26 monoclonal antibody 56 VL
DIQMTQSPSSLSASVGDRVTITCRASQSISNYLNWYQQKPGKAPKLLIYVASSLQSGVPSRFS
GSGSGTDFTLTISSLQPEDFATYYCQQSYSTPFTFGPGTKVDIK
SEQ ID NO. 27 DIA-23 sequence
QITLKESGPTLVKPTQTLTLTCTFSGFSLSTSGVGVAWIRQPPGKALEWLALIYWDNDKRSSP
SLNNRLTITKDTSKNQVVLTMTNMDPEDTATYYCAHFFSHYDSSNYYYGSWFDPWGQGTLV
TVSSGGGGSGGGGSDIQMTQSPSSLSASVGDRVTITCRASQSISNYLNWYQQKPGKAPKLLI
YVASSLQSGVPSRFSGSGSGTDFTLTISSLQPEDFATYYCQQSYSTPFTFGPGTKVDIKGGG
GSGGGGSGGGGSGGGGSGGGGSEVQLVESGGGVVQPGGSLRLSCAVSGFTFDDYAMHW
VRQAPGKGLDWVSLISGDGSYTYYADSVKGRFTISRDSSKNSLYLQMNSLRTEDTALYYCAK
AQTPTLWWLQDAFDIWGQGTMVTVSSGGGGSGGGGSEIVLTQSPGTLSLSPGERATLSCRA
SQSFDSRYLGWYQQKSGQAPRLLIYGASSRATGIPDRFSGSGSGTDFTLTISRLEPEDFAVYY
CQQFGDSPFTFGQGTKLEIKGGGGSDKTHTCPPCPAPEAAGGPSVFLFPPKPKDTLYITREP
EVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKS
LSLSPGK

All publications mentioned in the present disclosure are incorporated in the present application by reference to the same extent as if each individual publication was individually indicated to be incorporated by reference. Furthermore, it should be understood that, after reading the above teachings of the present disclosure, those skilled in the art can make various changes or modifications to the present disclosure, and these equivalent forms shall also fall within the scope defined by the claims attached to the present application.

1. A bispecific antibody against novel coronaviruses, comprising:
a first targeting domain D1 that binds to a first epitope on a Receptor Binding Domain (RBD) of a SARS-CoV-2 Spike (S) protein; and
a second targeting domain D2 that binds to a second epitope on the RBD of the SARS-CoV-2 S protein;
wherein the first epitope of the SARS-CoV-2 RBD domain comprises one or more residues from the group consisting of positions: 346R, 437N, 438S, 439N, 440N, 441L, 443S, 444K, 445V, 446G, 447G, 448N, 449Y, 450N, 498Q, 499P, 500T, and 506Q; and
the second epitope of the SARS-CoV-2 RBD domain comprises one or more residues from the group consisting of positions: 352A, 353W, 355R, 357R, 393T, 394N, 396Y, 462K, 463P, 464F, 465E, 466R, 468I, 516E, 518L, 519H, and 520A.

2. The bispecific antibody of claim 1, wherein:
the first targeting domain D1 comprises a first heavy chain variable region and a first light chain variable region, wherein
the first heavy chain variable region comprises a HCDR1 of SEQ ID NO: 11, a HCDR2 of SEQ ID NO: 12 and HCDR3 of SEQ ID NO: 13, and
the first light chain variable region comprises a LCDR1 of SEQ ID NO: 14, a LCDR2 of SEQ ID NO: 15, and a LCDR3 of SEQ ID NO: 16; and
the second targeting domain D2 comprises a second heavy chain variable region and a second light chain variable region, wherein
the second heavy chain variable region comprises a HCDR1 of SEQ ID NO: 17, a HCDR2 of SEQ ID NO: 18, and a HCDR3 of SEQ ID NO: 19, and
the second light chain variable region comprises a LCDR1 of SEQ ID NO: 20, a LCDR2 of SEQ ID NO: 21 and a LCDR3 of SEQ ID NO: 22.

3. The bispecific antibody of claim 2, wherein the first heavy chain variable region has an amino acid sequence of SEQ ID NO: 23, and the first light chain variable region has an amino acid sequence of SEQ ID NO: 24; and the second heavy chain variable region has an amino acid sequence of SEQ ID NO: 25, and the second light chain variable region has an amino acid sequence of SEQ ID NO: 26.

4. The bispecific antibody of claim 1, wherein the bispecific antibody is formed by fusion of antigen-binding fragments of D1 and D2, comprising two symmetric pairs of peptide chains linked by a disulfide bond, wherein each pair of peptide chains has structures of formulas a to d from an N-terminus to a C-terminus: 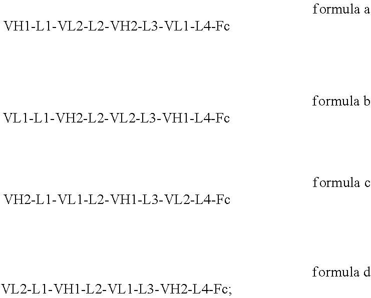
wherein
VH1 is the first heavy chain variable region, and VL1 is the first light chain variable region;
VH2 is the second heavy chain variable region, and VL2 is the second light chain variable region;
L1, L2, L3 and L4 are each independently selected from the group consisting of: absent, a bond, or a linker;
Fc is an Fc element;
“-” represents a peptide bond.

5. A polynucleotide, encoding the bispecific antibody of claim 1.

6. A vector, comprising the polynucleotide of claim 5.

7. A host cell, comprising (i) a genome incorporating the polynucleotide of claim 5; or (ii) a vector comprising the polynucleotide of claim 5.

8. A method for preparing the bispecific antibody of claim 1, comprising:
(i) culturing a host cell under conditions suitable for antibody expression, to obtain a mixture comprising the bispecific antibody of claim 1; and
(ii) purifying and/or separating the mixture obtained in step (i), to obtain the bispecific antibody of claim 1;
wherein the host cell comprises (i) a genome incorporating a polynucleotide encoding the bispecific antibody of claim 1; or (ii) a vector comprising the polynucleotide encoding the bispecific antibody of claim 1.

9. A pharmaceutical composition, comprising:
(I) the bispecific antibody of claim 1; and
(II) a pharmaceutically acceptable carrier.

10. (canceled)

11. An immunoconjugate, comprising:
(a) the bispecific antibody of claim 1; and
(b) a conjugated moiety selected from the group consisting of: a detectable marker, a drug, a toxin, a cytokine, a radionuclide, an enzyme, and combinations thereof.

12. A test reagent or kit for detecting a novel coronavirus infection, comprising
(a) the bispecific antibody of claim 1;
(b) a host cell comprising (i) a genome incorporating a polynucleotide encoding the bispecific antibody of claim 1, or (ii) a vector comprising the polynucleotide encoding the bispecific antibody of claim 1;
(c) a pharmaceutical composition comprising the bispecific antibody of claim 1 and a Pharmaceutically acceptable carrier; or
(d) an immunoconjugate comprising the bispecific antibody of claim 1 and a conjugated moiety;
wherein the novel coronavirus is one or more of a SARS-CoV-2 Prototype strain and a SARS-CoV-2 variant strain.

13. The bispecific antibody of claim 4, wherein the Fc element comprises amino acid substitutions selected from: L234A and L235A, and/or M252Y, S254T, and T256E.

14. The bispecific antibody of claim 4, wherein the bispecific antibody comprises an amino acid sequence of any one of SEQ ID NOs: 1 to 4 and 27.

15. The pharmaceutical composition of claim 9, wherein the pharmaceutical composition is in a form of nasal spray, oral preparation, suppository, or parenteral preparation.

16. The pharmaceutical composition of claim 9, wherein the pharmaceutical composition is a nasal spray.

17. The test reagent or the kit of claim 12, wherein the SARS-CoV-2 variant strain is selected from: SARS-CoV-2 Alpha (B.1.1.7), Beta (B.1.351), Gamma (P.1), Kappa (B.1.617.1), Delta (B.1.617.2) variant strains or Omicron (B.1.1.529) and subvariant strains thereof.

18. The test reagent or the kit of claim 12, wherein the novel coronavirus is SARS-CoV-2 selected from the group consisting of: prototype strains, and mutant strains BA.1, BA.1.1, BA.2, BA.2.12.1, BA.2.75, BA.3, BA.4, BA.5, BF.7, BQ.1, BQ.1.1, XBB, XBB.1.5, XBB.1.16, and EG.5.

19. A method for preventing or treating a novel coronavirus infection in a subject, comprising administering to the subject a therapeutically effective amount of
(a) the bispecific antibody of claim 1;
(b) a host cell comprising (i) a genome incorporating a polynucleotide encoding the bispecific antibody of claim 1, or (ii) a vector comprising the polynucleotide encoding the bispecific antibody of claim 1;
(c) a pharmaceutical composition comprising the bispecific antibody of claim 1 and a pharmaceutically acceptable carrier; or
(d) an immunoconjugate comprising the bispecific antibody of claim 1 and a conjugated moiety;
wherein the novel coronavirus is one or more of a SARS-CoV-2 prototype strain and a SARS-CoV-2 variant strain.

20. The method of claim 19, wherein the SARS-CoV-2 variant strain is selected from: SARS-CoV-2 Alpha (B.1.1.7), Beta (B.1.351), Gamma (P.1), Kappa (B.1.617.1), Delta (B.1.617.2) variant strains or Omicron (B.1.1.529) and subvariant strains thereof.

21. The method of claim 19, wherein the novel coronavirus is SARS-CoV-2 selected from the group consisting of: prototype strains, and mutant strains BA.1, BA.1.1, BA.2, BA.2.12.1, BA.2.75, BA.3, BA.4, BA.5, BF.7, BQ.1, BQ.1.1, XBB, XBB.1.5, XBB.1.16, and EG.5.
